(19) United States
(12) Patent Application Publication
(10) Pub. No.: US 2026/0201396 A1
(43) Pub. Date: Jul. 16, 2026

(54) New LoxPsym Sites for Large-Scale Orthogonal Cre-Mediated Recombination

(71) Applicants: VIB VZW, Gent (BE); Katholieke Universiteit Leuven, Leuven (BE)

(72) Inventors: Kevin Verstrepen, Leuven (BE); Charlotte Cautereels, Leuven (BE)

(21) Appl. No.: 19/115,408

(22) PCT Filed: Sep. 28, 2023
(86) PCT No.: PCT/EP2023/076902

§ 371 Date: Mar. 26, 2025

(30) Foreign Application Priority Data
Sep. 29, 2022 (EP) 22198794.4
Aug. 10, 2023 (EP) 23190947.4

(51) Int. Cl. C12N 15/81; C12N 15/70; C12N 15/82

(52) U.S. Cl. CPC C12N 15/81; C12N 15/70; C12N 15/815; C12N 15/8201; C12N 2800/30

(57) ABSTRACT

The invention relates to the field of genetic engineering and synthetic biology, more particularly to means and methods for facilitating genetic recombination. The application discloses novel Cre recombinase dependent recombination sites that supports a simultaneous cloning and testing approach.

### CROSS REFERENCE TO RELATED APPLICATIONS

This application is a continuation of U.S. patent application Ser. No. 16/757,033, filed Apr. 17, 2020, pending, which is a national phase entry under 35 U.S.C. § 371 of International Patent Application PCT/EP2023/076902, filed Sep. 28, 2023, designating the United States of America and published in English as International Patent Publication WO2024/068845 on Apr. 4, 2024, which claims the benefit under Article 8 of the Patent Cooperation Treaty to European Patent Application Serial No. 22198794.4, filed Sep. 29, 2022, and European Patent Application Serial No. 23190947.4, filed Aug. 10, 2023, the entireties of which are hereby incorporated by reference.

### INCORPORATION BY REFERENCE

The ST.26 XML Sequence listing named “10488-10901-US-SequenceListing”, created on Sep. 28, 2023, and having a size of 81,920 bytes, is hereby incorporated herein by this reference in its entirety.

### FIELD OF THE INVENTION

The invention relates to the field of genetic engineering and synthetic biology, more particularly to means and methods for facilitating genetic recombination. The application discloses novel Cre recombinase dependent recombination sites that support a simultaneous cloning and testing approach.

### BACKGROUND

Site specific recombination has proven to be a powerful tool in genetic engineering, developmental biology and systems biology over the past decades. Such recombination systems are very popular in several research fields, as they enable to delete, invert, integrate and translocate large chunks of genomic DNA in vivo in several host organisms (Meinke et al 2016 Chem Rev 20:12785-12820). Specifically, the Cre (Causes recombination) recombinase is one of the most commonly used recombination systems, as it is well-understood, it has been implemented and characterized in several inducible formats (Guo et al 2002 Genesis 32:8-18; Wu et al 2020 Nat Commun 11:3708; Hochrein et al 2018 Nat Commun 9:1931) and it is functional in a wide-range of host organisms (Adams 1992 J Mol Biol 226:661-73; Sauer 1987 Mol Cell Biol 7:2087-2096; Hoa et al 2002 Theor Appl Genet 104:518-525; Shimshek et al 2002 Genesis 32:19-26). The Cre recombinase, originally deriving from bacteriophage P1, acts via recognition of the LoxP site. This is a 34 bp long sequence existing of two 13 bp inverted repeats that flank a directional 8 bp spacer (Sternberg and Hoess 1983 Annu Rev Genet 17:123-154). During recombination, the Cre recombinase binds both inverted repeats of the LoxP site as a dimer, cuts the spacer region at both strands and initiates strand exchange with another LoxP site (Guo et al 2000 Genesis 32:8-18). Depending on the orientation and position of the LoxP site deletion, inversion or translocation of a DNA fragment can occur.

To enable non-directional recombination, which allows different recombination events to occur independently of the orientation of the recombination sites, the spacer of the LoxP site has been converted to a palindromic site by Hoess et al. (1986 Nucleic Acids Res 14:2287-2300). This artificial recombination site is referred to as LoxPsym and has recently profited from a newfound interest, as it plays a major role in the Synthetic Yeast Genome project—Sc 2.0. The latter aims to build the world's first synthetic eukaryotic genome whilst making it hyper-evolvable on demand. This is achieved via the introduction of thousands of LoxPsym sites across the S. cerevisiae genome, allowing rapid introduction of genomic rearrangements upon activation of the inducible Cre recombinase (Richardson et al 2017 Science 355:1040-1044).

Despite its broad application, the Cre-LoxPsym system is limited in its use to a single-recombination system. Therefore, several approaches have been made in the recent years to obtain orthogonal recombination systems within one organism. Orthogonality allows simultaneous, large-scale and independent gene recombination in different regions of the genome. Orthogonal recombination systems not only enable more highly sophisticated genome engineering in synthetic biology, they also have broad applications in other fields, such as developmental biology (Weng et al 2022 Trends Cell Biol 32:324-337), metabolic engineering (Liu et al 2017 Methods in Molecular Biology, vol 1642) and environmental monitoring (Akboğa et al 2022 Biosensors 12:122).

Currently, orthogonal recombination is obtained via combining multiple tyrosine recombinases, which act without cross-reactivity as they recognize distinct recombination sites. Several new tyrosine recombinases which work orthogonal to Cre-LoxP have been discovered in recent years. These include Vcre (Liu et al 2018 Nat Commun 9:1936), SCre (Suzuki et al. 2011 Nucleic Acid Res 39: e49) and Vika (Karimova et al. 2013 Nucleic Acid Res 42: e37). Recently, also the development of orthogonal non-directional recombination systems for Vika and Dre have been published (Wang et al. 2021 iScience 25:103716). However, the number of non-cross-reacting recombinases remains limited and using several recombination systems within a new host organism requires heterologous expression of various enzymes, potentially toxic to the host. To tackle these shortcomings, we build further on existing knowledge and developed another strategy to obtain orthogonal recombination, by developing non-cross-reacting LoxPsym recombination sites.

### SUMMARY

In current application, it is disclosed how the inventors first characterized 63 new LoxPsym sites by editing the spacer of the LoxPsym site. Several sites showed a higher recombination efficiency compared to the commonly used LoxPsym site. Afterwards, they performed an intensive screening to identify LoxPsym variants which can act without cross-reactivity and found that several sets of orthogonal LoxPsym variants can be made.

In a first aspect, the application provides a LoxPsym site having the following formula:
5′-ATAACTTCGTATA—spacer—TATACGAAGTTAT, wherein the spacer is selected from the list consisting of SEQ ID No. 1-63. In one embodiment, the LoxPsym site is cleaved in the presence of recombinase Cre. In another embodiment, the LoxPsym site comprises or consists of the nucleic acid sequence as depicted in SEQ ID No. 65-127. In one particular embodiment, the LoxPsym site is characterized by a recombination efficiency that is lower than that of the standard LoxPsym site as depicted in SEQ ID No. 128, wherein the LoxPsym site comprises a spacer selected from the list consisting of SEQ ID No. 1-27. In another particular embodiment, the LoxPsym site is characterized by a recombination efficiency that is higher than that of the standard LoxPsym site as depicted in SEQ ID No. 128, wherein the LoxPsym site comprises a spacer selected from the list consisting of SEQ ID No. 28-63. In another particular embodiment, the LoxPsym site is an orthogonal LoxPsym site, meaning that in the presence of recombinase Cre, a specific DNA recombination cannot occur between said LoxPsym site and another LoxPsym site comprising a different nucleotide on position 2, 3, 6 and/or 7 of the spacer. In a particular embodiment, the LoxPsym site and the other LoxPsym site do not comprise the spacers GGGTACCC and AACTAGTT, GGGTACCC and ATATATAT, GAATATTC and AGTTAACT, GAGTACTC and AAATATTT or GTGTACAC and GCATATGC respectively. In a most particular embodiment, the LoxPsym site is selected from the list consisting of SEQ ID No. 65, 69-71, 74, 77, 82, 84, 100, 105, 107-109, 112, 121 and/or 124. Also a vector is provided comprising any of the LoxPsym sites herein described as well as host cell comprising the vector. The host cell can be a plant cell, a bacterial cell, a yeast cell, an insect cell or a mammalian cell.

In a second aspect, a set of at least two LoxPsym sites are provided, the LoxPsym sites comprise or consist of the following formula:
5′-ATAACTTCGTATA—spacer—TATACGAAGTTAT, wherein the spacer is selected from the list consisting of SEQ ID No. 1-63, and wherein the at least two LoxPsym sites comprise a different nucleotide on position 2, 3, 6 and/or 7 of the spacer, and wherein the set does not comprise the LoxPsym sites depicted in SEQ ID No. 88 and 89, SEQ ID No. 88 and 81, SEQ ID No. 90 and 83, SEQ ID No. 65 and 92, or SEQ ID No. 78 and 103. In a particular embodiment, the at least two LoxPsym sites are selected from SEQ ID No. 65, 69-71, 74, 77, 82, 84, 100, 105, 107-109, 112, 121 and/or 124.

In a third aspect, a vector comprising one or more LoxPsym sites according the first aspect of the invention is provided. In a particular embodiment, said vector comprises the set of at least two LoxPsym sites according to any embodiment of the second aspect of the invention.

In another aspect a host cell comprising one or more of the LoxPsym sites according the first aspect of the invention or the vector according to the third aspect of the invention is provided. In a particular embodiment, the host cell comprises the set of at least two LoxPsym sites according to the second aspect.

In another aspect, the use of any of the LoxPsym sites or vectors or of sets of LoxPsym sites herein disclosed are provided for site-specific recombination of one or more nucleic acid sequences. Also the use of these LoxPsym sites, sets of LoxPsym sites and/or vectors is provided for in vivo cloning and phenotyping. In a particular embodiment, said cloning and phenotyping is done sequentially in the same cell.

In yet another aspect, a method for obtaining a recombinant nucleic acid molecule is provided, the method comprises the steps of: i) providing a nucleic acid molecule comprising two or more nucleic acid elements each flanked by an orthogonal LoxPsym site or providing a first and a second nucleic acid molecule each comprising one or more nucleic acid elements, the one or more nucleic acid elements each flanked by an orthogonal LoxPsym site; and ii) reacting the nucleic acid molecule or the first and second nucleic acid molecules with recombinase Cre to obtain a recombinant nucleic acid molecule, wherein the orthogonal LoxPsym site comprises or consist of any of SEQ ID No. 65-127.

Also provided is a method for shuffling DNA elements within a nucleic acid molecule, the method comprises the steps of: i) providing a nucleic acid molecule comprising at least two nucleic acid elements individually flanked by an orthogonal LoxPsym site; and ii) reacting the nucleic acid molecule with recombinase Cre, to obtain a nucleic acid molecule in which the nucleic acid elements are reshuffled, wherein the orthogonal LoxPsym site comprising or consisting any of SEQ ID No. 65-127.

In one embodiment of the methods, the orthogonal LoxPsym site is site is selected from the list consisting of SEQ ID No. 65, 69-71, 74, 77, 82, 84, 100, 105, 107-109, 112, 121 and/or 124. In another embodiment, the methods further comprise a step of determining the sequence of the recombinant nucleic acid molecule. In yet another embodiment, the methods further comprise the step of introducing the recombinant nucleic acid molecule in a cell and/or determining the expression of the recombinant nucleic acid molecule in a cell. Also provided is a recombinant nucleic acid molecule obtained by the methods herein described.

In a final aspect, a method of optimizing gene expression of one or more genes in a cell is provided, comprising the steps of: i) expressing or introducing in a cell, one or more vectors comprising the one or more genes, each gene being under control of a promoter, the promoter comprising two or more promoter elements, wherein the two or more promoter elements are individually flanked by an orthogonal LoxPsym site, wherein a different LoxPsym site is used per gene; and ii) optionally, the one or more vectors further comprise a terminator sequence downstream of each gene, the terminator sequence comprising two or more terminator elements, wherein the two or more terminator elements are individually flanked by an orthogonal LoxPsym site, wherein a different LoxPsym site is used per gene and wherein any of the LoxPsym sites from step ii) are different to any of the LoxPsym sites used in step i); and iii) expressing in said cell a recombinase Cre; and iv) analyzing the gene expression of the one or more genes or analyzing the phenotype of the cell. In one embodiment, the at least one orthogonal LoxPsym site is a LoxPsym site selected from any of the LoxPsym sites herein disclosed, more particularly selected from the list consisting of SEQ ID No. 65, 69-71, 74, 77, 82, 84, 100, 105, 107-109, 112, 121 and/or 124. In another embodiment, the method further comprises the step of determining the sequence of the whole or part of the cell's genome. Also provided is a cell, particularly a genetically engineered cell, obtained by the method of optimizing gene expression as described herein. In one embodiment, the cell is a plant cell, a bacterial, cell, a yeast cell, an insect cell or a mammalian cell.

Introducing one or more vectors in a cell is the same as genetically transforming a cell with one or more vectors. The skilled person is familiar with a plethora of molecular technique to perform such transformation.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

FIG. 1 is a schematic representation of the genetic expression optimization tool herein disclosed, which consists out of a promoter (upstream the GOI) and terminator (downstream the GOI) construct driving the expression of a gene of interest (GOI shown in grey). Orthogonal LoxPsym sites flanking the promoter elements (6 promoter elements are shown as example without the intension of being limited) are visualized by diamonds colored in green), while the LoxPsym sites flanking the terminator elements (6 terminator elements are shown as example without the intension of being limited) are visualized by circles colored in yellow. The promoter and terminator elements have different strengths as indicated by different shades of blue. Upon induction of Cre mediated recombination, genetic rearrangements of both constructs will randomly enable different parts of the constructs to drive the expression of the gene of interest, which will result in different expression levels and thus in phenotypic variation within the yeast population (represented by differently colored yeast cells), of which some recombinants will have obtained superior phenotypic traits. The expression optimization tool can simultaneously be applied on multiple genes of a pathway.

FIG. 2 is an illustration of the LoxPsym site or sequence and how variants are obtained by adapting the first three nucleotides of the spacer, shown in grey, and changing the rest of the spacer accordingly to obtain a palindromic LoxPsym site. The Cre recombinase as illustrated by the green structure cuts the LoxPsym site in the spacer region at both strands.

FIGS. 3A-3I: FIG. 3A shows the genetic construct that was used for determining the deletion/inversion frequency caused by Cre-LoxPsym recombination, integrated at the CAN1 locus.

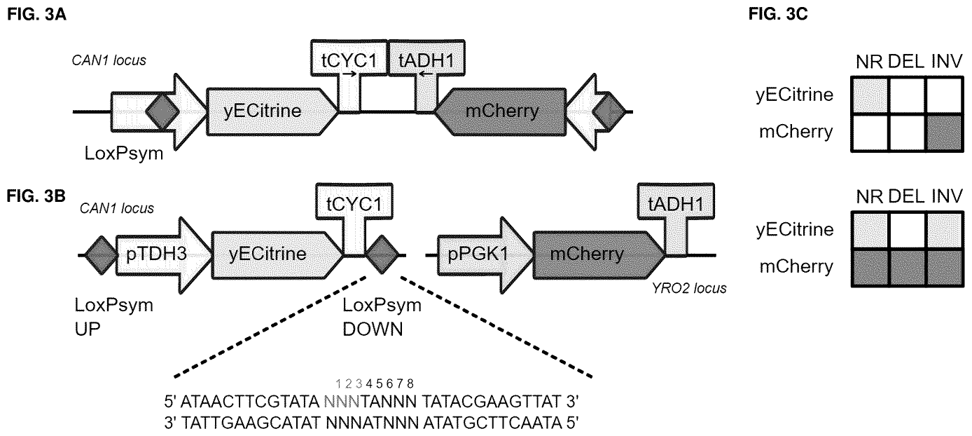

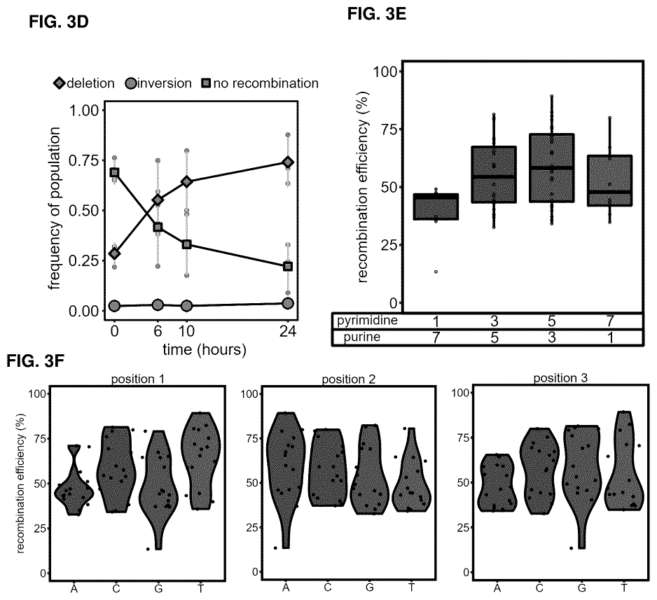

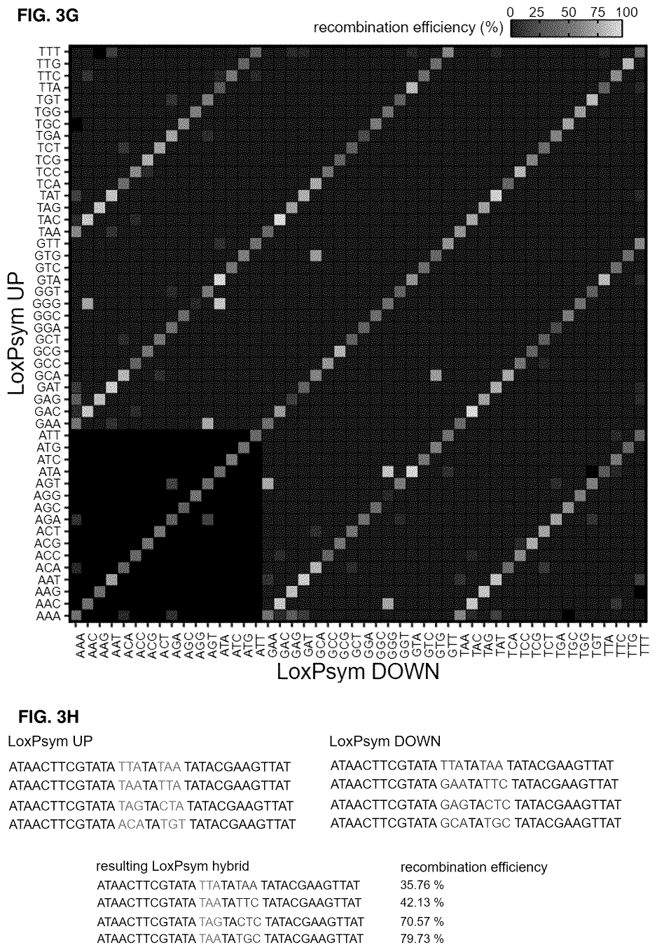

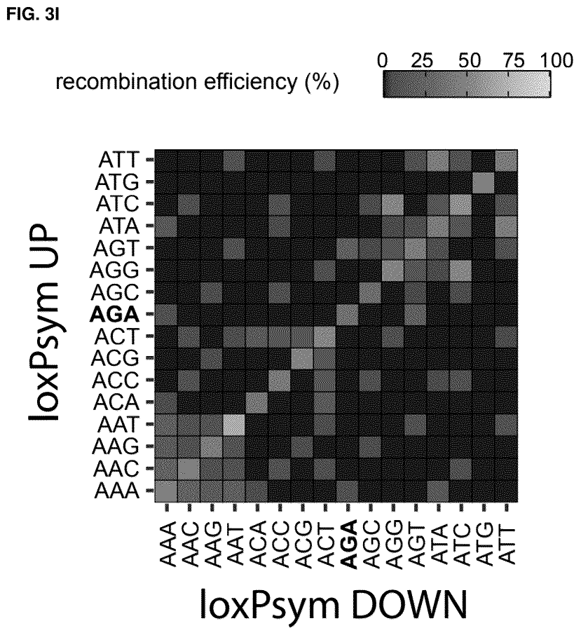

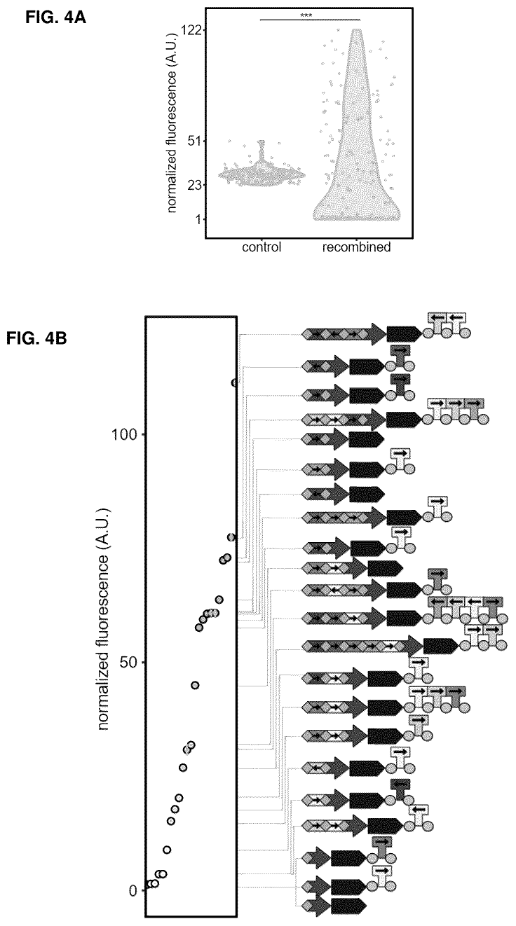

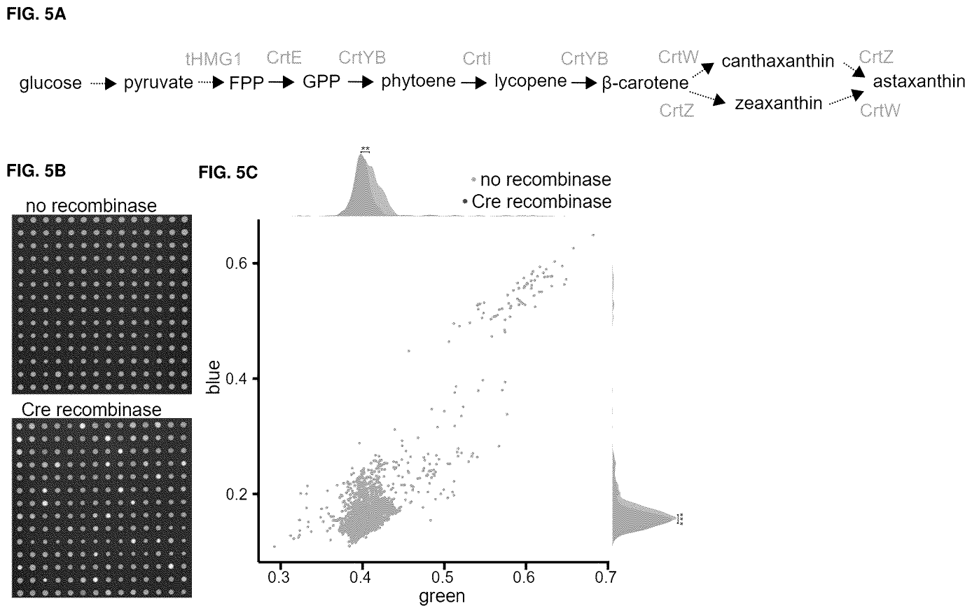

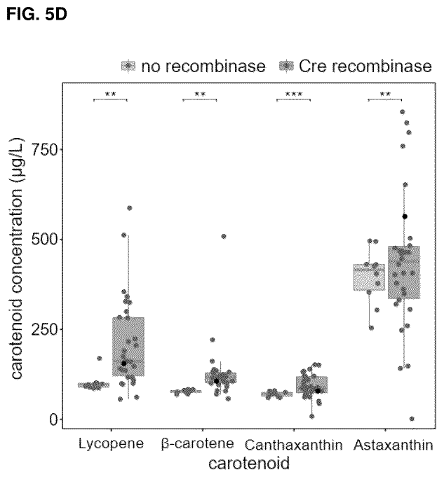

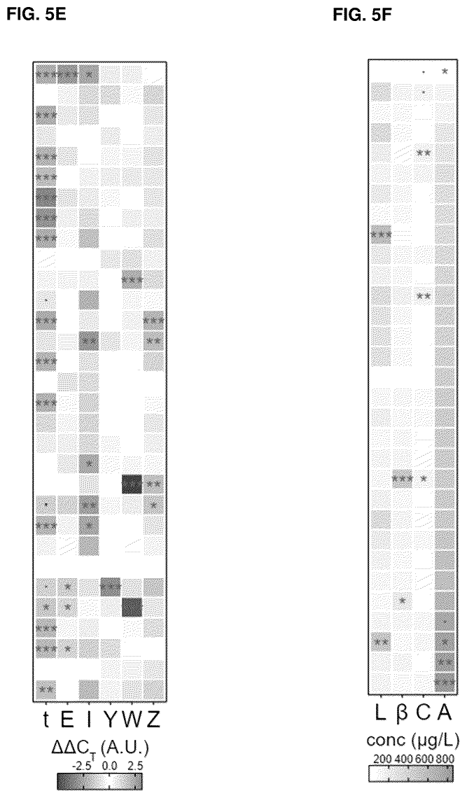

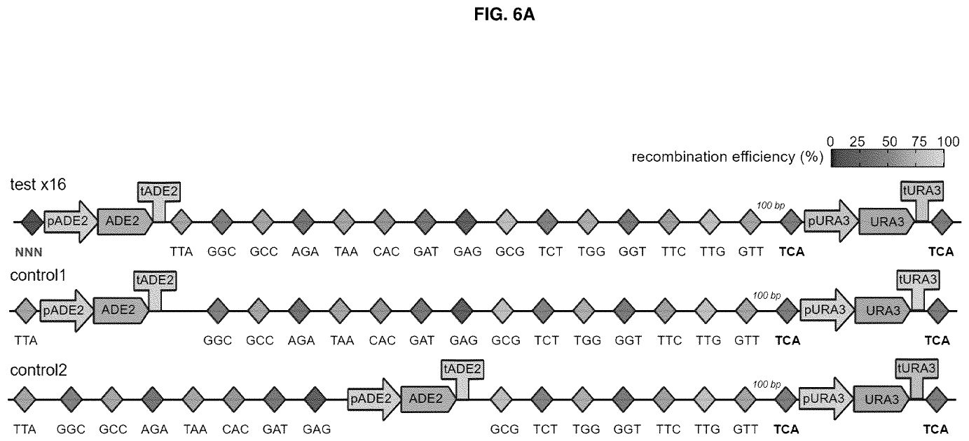

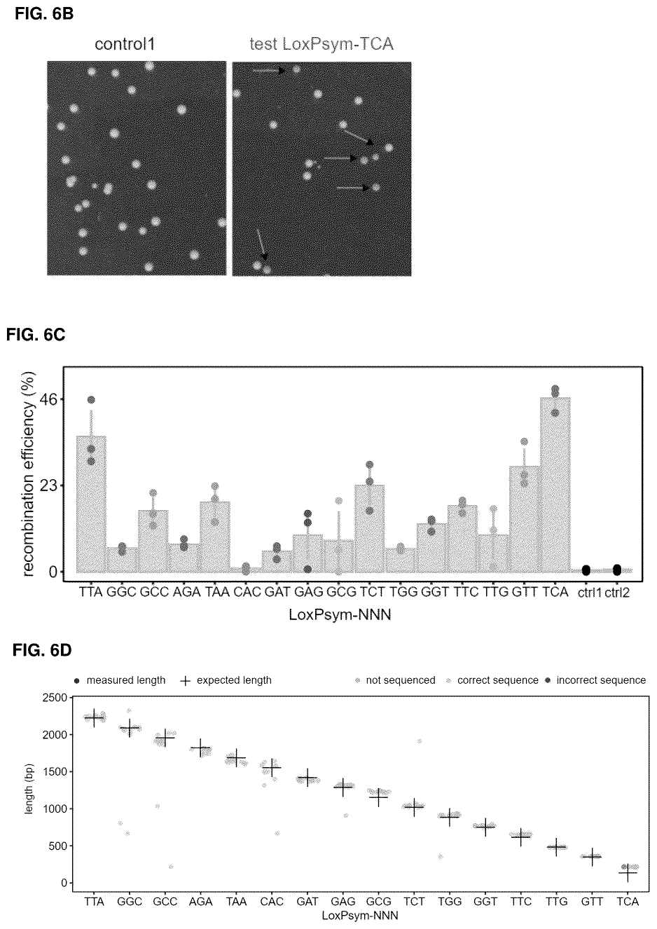

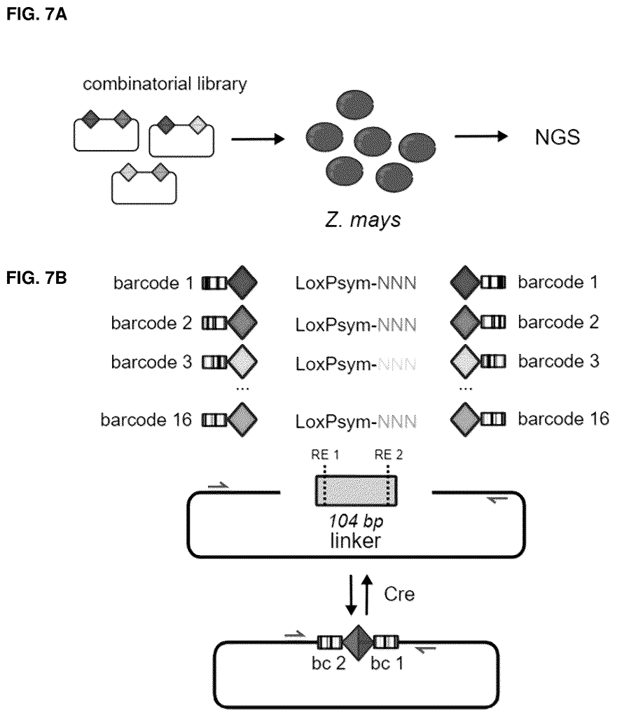

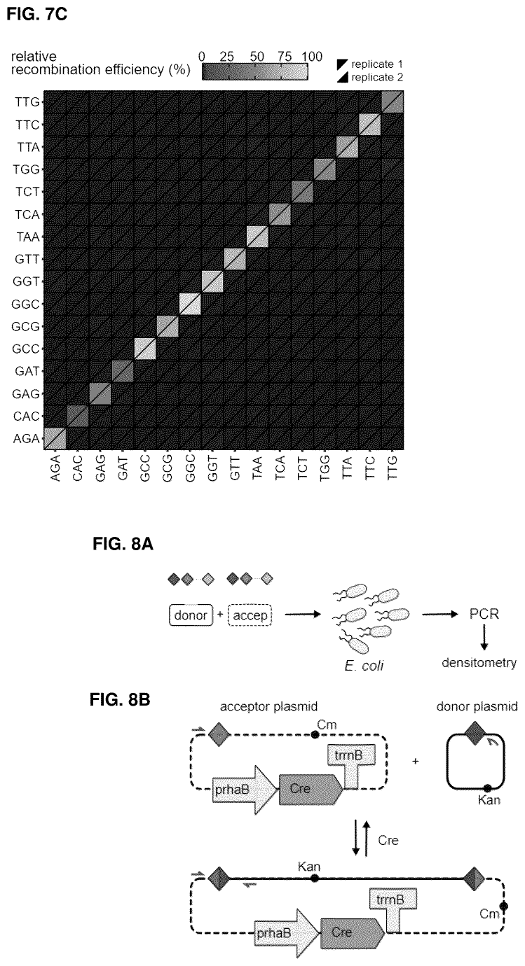

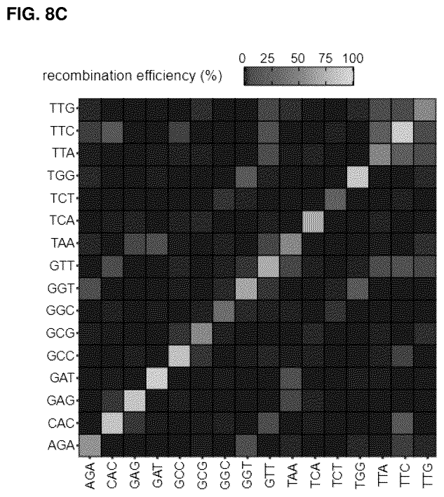

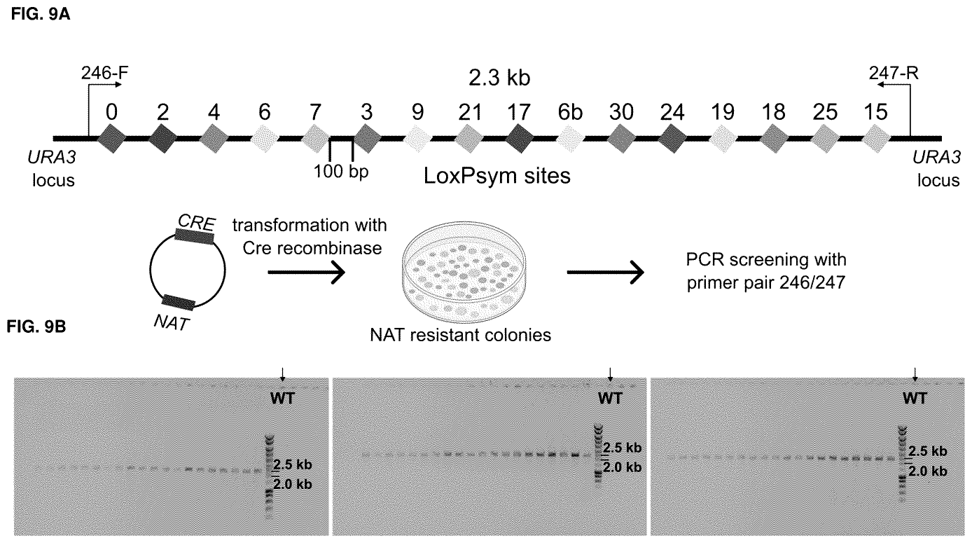

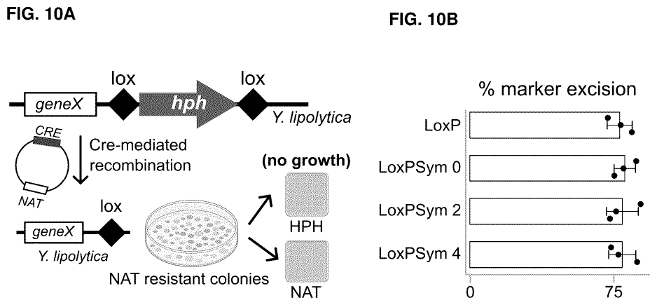

LoxPsym (green diamond) is placed in the TDH3 promoter (yellow arow) directly in front of the core promoter. This layout results in yECitrine fluorescence and prevents mCherry fluorescence. FIG. 3B shows the genetic construct that was used for determining the Cre-LoxPsym recombination efficiency. 64 LoxPsym variants are tested by altering base pairs at position 1, 2 and 3 of the spacer (blue). LoxPsym sites flank high expression cassette pTDH3-yECitrine-tCYC1, integrated at CAN1 locus. Expression cassette pPGK1-mCherry-tADH1 is integrated at the YRO2 locus, serving as a control. FIG. 3C is a schematic representation of fluorescence in the cells after induction of the Cre recombinase, resulting in no recombination (NR), deletion (DEL) or inversion (INV) of the fluorescence cassette shown in panel a (top) and b (bottom). Yellow, red and white colors indicate yECitrine, mCherry or no fluorescence respectively. FIG. 3D shows the frequency of the population with deletion (diamonds), inversion (circle) or original (square) fluorescence cassette shown in panel a, tracked over a time course of 24 h. FIG. 3E shows the recombination efficiency grouped by the number of purines/pyrimidines in the spacer sequence. FIG. 3f shows the recombination efficiency grouped by the nucleotide at position 1, 2 or 3. FIGS. 3G and 3I show the recombination efficiency, determined from the cassette shown in panel b, between 48 LoxPsym sites after 6 h induction of Cre expression.

The LoxPsym variants are characterized by the nucleotides at position 1, 2 and 3 of their spacer sequence. Hence, AAA stands for spacer AAATATTT and AAC for spacer AACTAGTT, etc. FIG. 3H shows the resulting LoxPsym sequence after deletion of the reporter gene.

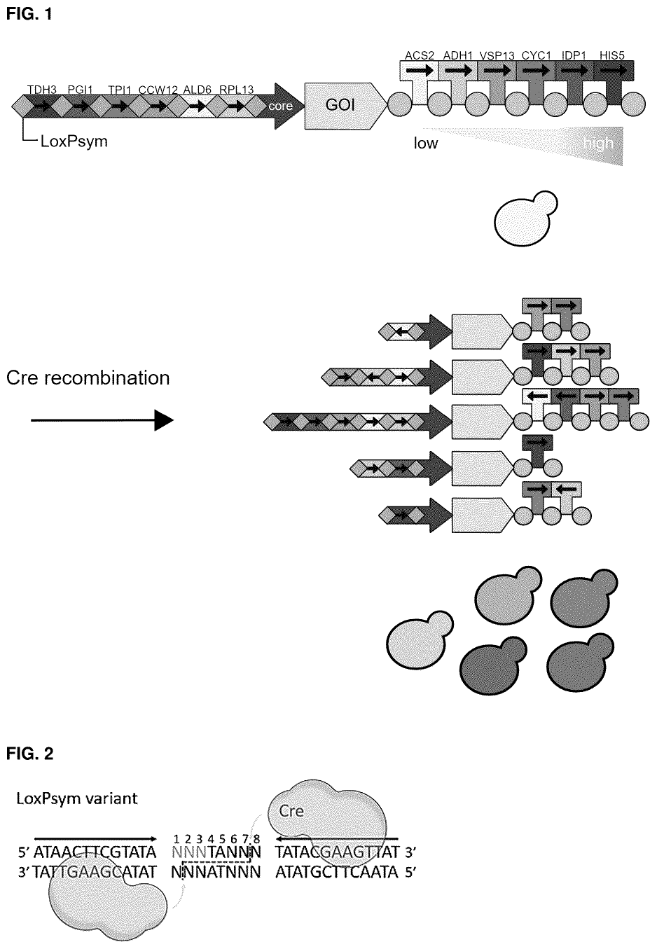

FIGS. 4A and 4B: FIG. 4A shows a comparison of normalized fluorescence of single SCRaMbLEants between a control group (grey) and a Cre-expressing group (yellow), bearing a yECitrine gene regulated by the promoter and terminator constructs shown in FIG. 1 (N=180). Dots represent normalized fluorescence of separate SCRaMbLEants. Statistics by Fligner-Killeen with p-value 2.20E-16. Green dots indicate SCRaMbLEants which were further analyzed and sequenced, shown in panel b. FIG. 4b shows the normalized fluorescence upon recombination of promoter and terminal elements. Dots represent average of 3 biological repeats, grey error bars represent standard deviation. The sequences of the recombined promoter and terminator constructs are depicted on the right of each graph, using a grey line to indicate their fluorescence.

FIGS. 5A-5F: FIG. 5A is a schematic representation of the astaxanthin production pathway in S. cerevisiae. Heterologous genes which were targeted for expression optimization are indicated in orange. Arrows indicate enzymatic conversion of molecules and dashed lines indicate multiple intermediate steps underlie this conversion. FIG. 5B shows single picked colonies after SCRaMbLE was induced. Large color variation in the Cre+ strains was obtained compared to the strains without the recombinase. FIG. 5C shows the quantification of the RGB-value of 1408 and 1490 single clones from the Cre+ and Cre− groups respectively. Statistics by Kligner-Killeen with p-value 3.23E-03 and 8.30E-10 across the x- and y-axis respectively. FIG. 5D shows the carotenoid titers obtained by LC-MS analysis of aceton extracts, harvested from cultures which were grown for 72 h in 50 ml YPD2%. Cre+ strains (N=30, right) and strains without a recombinase (N=10, left). FIG. 5E shows the ΔΔCT values obtained from qPCR analysis by comparison with the unaltered clone B, indicating the gene expression level of each of the 6 studied genes (tHMG, CrtE, CrtI, CrtYB, CrtW and CrtZ) of the astaxanthin production pathway. FIG. 5F shows the metabolite concentrations (μg/L) of the single clones.

FIGS. 6A-6D: FIG. 6 shows limited cross-reactivity between 16 LoxPsym variants simultaneously in S.cerevisiae. FIG. 6A shows in total 18 constructs, which were designed: 16 test and 2 control constructs. Each construct included all 16 different LoxPsym variants tested, as well as an ADE2 and URA3 expression cassette. The URA3 cassette was in every case flanked by LoxPsym-TCA and served as a control to ensure active, functional recombination. The controls tested 2 locations of the ADE2 cassette-upstream and surrounded by the LoxPsym-array- and should not result in ADE2 deletion if the 16 LoxPsym variants operate orthogonally. The 16 test constructs differed in the LoxPsym-NNN variant upstream of ADE2. The different test constructs verified cross-reactivity between all sites as well as the activity and specificity of recombination between the identical LoxPsym-NNN pairs. The LoxPsym variants in the array were separated by 100 bp, allowing recombination between adjacent sites. All constructs were inserted at the CAN1 locus of BY4741 AADE2. To induce recombination, all strains were transformed with plasmid pSH47-His-Cre or the negative control pSH47-His-Vec. FIG. 6B shows that after 6 h induction, the cells were plated on SC+FOA plates to select for URA3 deletion caused by recombination of the canonical LoxPsym sites surrounding the URA3 marker. Red clones from the test strains indicating a deletion of the ADE2 cassette were selected for further investigation via PCR and sequencing (marked with arrows). FIG. 6C shows the recombination efficiency (calculated from ADE2 deletion occurrence, red phenotype) representing plate counts of three biological replicates, error bars indicate standard deviation. The control strains showed negligible recombination efficiencies (0.4975±0.3518 and 0.5962±0.4268 for control 1 and 2, respectively). No colonies were observed for strains carrying pSH47-HisVec. FIG. 6D shows measured and expected length of the recombined construct of thirteen randomly picked red clones from each strain, represented by dots and crosses respectively. Three randomly picked samples were analyzed by Sanger sequencing; the different grey colors of the dot indicate the sequencing result.

FIGS. 7A-7C: FIG. 7 shows recombination and cross-reactivity of LoxPsym variants in Zea mays. FIG. 7A shows experimental design for determining LoxPsym variant cross-reactivity in Z. mays. The combinatorial library that included all 256 pairwise combinations of LoxPsym variants was transfected to Z. mays protoplasts together with a plasmid for constitutive expression of Cre. The presence of recombination was verified using NGS. FIG. 7B shows the design of the combinatorial library that was transfected to plant protoplasts. Each plasmid encoded two LoxPsym variants (variants are represented by differently shaded grey diamonds), separated by a 104 bp linker that contains recognition sites (RE1 and RE2) for restriction enzymes NcoI-HF and PvuI-HF (dashed lines). Barcodes were incorporated up- and downstream of the LoxPsym variants, with each barcode uniquely linked to one LoxPsym variant. The library contained all 16×16 (=256) combinations between LoxPsym variants. Arrows indicate primer annealing sites for the PCR that was carried out after recombination was induced. PCR products were analyzed for size on agarose gel and short amplicons (indicating recombination occurred) were analyzed by next generation sequencing FIG. 7C shows recombination efficiencies between LoxPsym-NNN variants in Z. mays, calculated from the abundance of sequenced reads. Note that all efficiencies were normalized to the most active recombination site, LoxPsym-GGC, for which the efficiency was arbitrarily set to 100%. Data represent the average of three technical repeats for two biological replicates shown separately by diagonally split cells.

FIGS. 8A-8C: FIG. 8 shows recombination and cross-reactivity of LoxPsym variants in Escherichia coli. FIG. 8A shows experimental design for determining LoxPsym variant cross-reactivity in E. coli. Two plasmids, a donor (full line) and acceptor (dashed line), were co-transformed. Both plasmids carry one LoxPsym variant (different shades of diamonds) and in vivo recombination was verified using PCR.

FIG. 8B shows details on donor and acceptor plasmid. The acceptor plasmid encodes the Cre gene, controlled by the rhamnose inducible rhaB promoter and rrnB terminator. Induction of Cre expression results in recombination if LoxPsym variants are cross-reactive. PCR (indicated by small arrows) was used to amplify the junction of recombined plasmids. Recombination was induced for 4 h by growing cells in LB supplemented with 2% rhamnose, experimental conditions based on previous reports (for example in Sheets 2020, Ceroni 2018). Note that the recombination reaction does not have a final state because recombined plasmids can recombine back to two separate plasmids. FIG. 8C shows recombination efficiencies between LoxPsym-NNN variants in E. coli, calculated from densitometric analysis of the junction PCR. Data represent band intensity of a PCR performed in technical duplicate with a mixture of templates derived from three biological replicates.

FIGS. 9A and 9B: FIG. 9 shows that alternative LoxPsym sites are orthogonal in Yarrowia lipolytica. FIG. 9A shows a schematic representation of the workflow proving that the alternative lox sites are orthogonal in Y. lipolytica. First a construct with the 16 LoxPSym sites (see Table 2) separated by 100 bp spacer sequences was integrated at the URA3 locus of Y. lipolytica (strain W29). The strain carrying the lox sites was then transformed with a plasmid carrying the Cre recombinase and the NAT selection marker, for the transient expression of the recombinase in Y. lipolytica. After the transformation the NAT resistant colonies were screened with the primer pair 246-F/247-R to validate that the different lox sites are orthogonal in Y. lipolytica. FIG. 9B shows a gel electrophoresis after the PCR screening of randomly selected transformants with the primer pair 246-F/247-R. From all the independent transformants a single band (2.3 kb) was amplified indicating that no recombination has occurred between the different lox sites. An additional PCR reaction with genomic DNA from the wild type non-transformed strain (indicated as WT at the gels) was served as a negative control.

FIGS. 10A and 10B: FIG. 10 shows recombination and cross-reactivity of LoxPsym variants in Y. lipolytica. FIG. 10A is a schematic representation of the use of the orthogonal lox sites for marker excision in Y. lipolytica. The gene of interest (gene X) has been integrated at a genomic region of Y. lipolytica adjacent to a selectable marker (hph), flanked by two putative orthogonal sites. The strain with this construct is then transformed with a plasmid carrying the Cre recombinase and the selectable marker NAT. The NAT resistant colonies are then transferred to fresh plates containing either the NAT or the HPH marker. If the sites are orthogonal and the marker is successfully excised no growth will be observed at the HPH selection plates. FIG. 10B shows three different LoxPsym sites which were tested for their efficiency to recombine, after Cre expression, and thus lead to marker excision. Removal of the hph marker was achieved after the transformation with the plasmid carrying the Cre recombinase. After the transformation 192 NAT resistant colonies were transferred to plates supplemented with hygromycin B and the growth of the transferred colonies was evaluated after 3 days. The frequency of marker excision was determined by calculating how many colonies out of the 192 lost their ability to grow on plates with hygromycin B. The experiments were performed in triplicates, and the bars indicate the SD between the replicates. For comparison, the same experiment was performed using the traditional LoxPsym (LoxP) sites.

### DETAILED DESCRIPTION

### Definitions

In order that the present description can be more readily understood, certain terms are first defined. Additional definitions are set forth throughout the detailed description. The present invention is described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. It is to be noted that the term “a” or “an” entity refers to one or more of that entity; for example, “a nucleotide sequence”, is understood to represent one or more nucleotide sequences. As such, the terms “a” (or “an”), “one or more” and “at least one” can be used interchangeably herein. Furthermore, “and/or” where used herein is to be taken as specific disclosure of each of the two specified features or components with or without the other. Thus, the term “and/or” as used in a phrase such as “A and/or B” herein is intended to include “A and B”, “A or B”, “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone). Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

It is understood that wherever aspects or embodiments are described herein with the language “comprising”, otherwise analogous aspects or embodiments described in terms of “consisting of” and/or “consisting essentially of” are also provided. Where the term “comprising” is used in the present description and claims, it does not exclude other elements or steps. Unless specifically defined herein, all terms used herein have the same meaning as they would to one skilled in the art of the present invention.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure. Practitioners are particularly directed to Sambrook et al., Molecular Cloning: A Laboratory Manual, 4th ed., Cold Spring Harbor Press, Plainsview, New York (2012); and Ausubel et al., current Protocols in Molecular Biology (Supplement 100), John Wiley & Sons, New York (2012), for definitions and terms of the art. The definitions provided herein should not be construed to have a scope less than understood by a person of ordinary skill in the art.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

As used herein, the terms “nucleic acid”, “nucleic acid sequence” or “nucleic acid molecule” are used interchangeably and refer to a polymeric form of nucleotides of any length, either deoxyribonucleotides or ribonucleotides, or analogs thereof. Nucleic acids may have any three-dimensional structure, and may perform any function, known or unknown. Non-limiting examples of nucleic acids include a gene, a gene fragment, exons, introns, a promoter or fragment thereof, a terminator or fragment thereof, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular. The nucleic acid may comprise a promoter, an intron, an enhancer region, a polyadenylation site, a translation initiation site, 5′ or 3′ untranslated regions, a reporter gene, a selectable marker or the like. The nucleic acid may comprise single stranded or double stranded DNA or RNA. The nucleic acid may comprise modified bases or a modified backbone. A nucleic acid that is up to about 100 nucleotides in length, is often also referred to as an oligonucleotide. “Nucleotides” as used herein refer to the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which are absent in nucleosides). A nucleotide without a phosphate group is called a “nucleoside” and is thus a compound comprising a nucleobase moiety and a sugar moiety. As used herein, “nucleobase” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Naturally occurring nucleobases of RNA or DNA comprise the purine bases adenine (A) and guanine (G), and the pyrimidine bases thymine (T), cytosine (C) and uracil (U).

The term “defined by SEQ ID No. X” or “as depicted in SEQ ID No. X” as used herein refers to a biological sequence consisting of the sequence of nucleotides given in the SEQ ID No. X. For instance, a LoxPsym site defined in/by SEQ ID No. X consists of the nucleic acid sequence given in SEQ ID No. X. A further example is a nucleic acid sequence comprising SEQ ID No. X, which refers to a nucleic acid sequence longer than the nucleic acid sequence given in SEQ ID No. X but entirely comprising the nucleic acid sequence given in SEQ ID No. X, or to a nucleic acid sequence consisting of the nucleic acid sequence given in SEQ ID No. X.

A “chimeric gene”, “chimeric gene construct” or “chimeric construct” is a recombinant nucleic acid sequence in which a promoter or regulatory nucleic acid sequence is operably linked to, or associated with, a nucleic acid sequence that codes for a mRNA and encodes an amino acid sequence, such that the regulatory nucleic acid sequence is able to regulate transcription or expression of the associated nucleic acid coding sequence. The regulatory nucleic acid sequence of the chimeric gene is not operably linked to the associated nucleic acid sequence as found in nature.

A “promoter” is a DNA sequence comprising regulatory elements, which mediate the expression of a nucleic acid molecule. For expression, the nucleic acid molecule must be linked operably to or comprise a suitable promoter which expresses the gene at the right point in time and with the required spatial expression pattern. The term “operably linked” as used herein refers to a functional linkage between the promoter sequence and the gene of interest, such that the promoter sequence is able to initiate transcription of the gene of interest. A promoter that enables the initiation of gene transcription in a eukaryotic or host cell is referred to as being “active”. To identify a promoter which is active in a eukaryotic or host cell, the promoter can be operably linked to a reporter gene after which the expression level and pattern of the reporter gene can be assayed. Suitable well-known reporter genes include for example beta-glucuronidase, beta-galactosidase or any fluorescent or luminescent protein. The promoter activity is assayed by measuring the enzymatic activity of the beta-glucuronidase or beta-galactosidase. Alternatively, promoter strength may also be assayed by quantifying mRNA levels or by comparing mRNA levels of the nucleic acid, with mRNA levels of housekeeping genes such as 18S rRNA, using methods known in the art, such as Northern blotting with densitometric analysis of autoradiograms, quantitative real-time PCR or RT-PCR (Heid et al., 1996 Genome Methods 6:986-994).

The term “a 3′ end region involved in transcription termination or polyadenylation” encompasses a control sequence which is a DNA sequence at the end of a transcriptional unit which signals 3′ processing or polyadenylation of a primary transcript and is involved in termination of transcription.

The control sequence for transcription termination or terminator can be derived from a natural gene or from a variety of genes. For expression in yeast the terminator to be added may be derived from, for example, the TEF or CYC1 genes or alternatively from another yeast gene or less preferably from any other eukaryotic or viral gene.

The term “vector” refers to any linear or circular DNA construct comprising one of the LoxPsym sites of the application. The vector can refer to an expression cassette or any recombinant expression system for the purpose of expressing a gene of interest in vitro or in vivo, constitutively or inducibly, in any cell, including yeast, plant and mammalian cells. The vector can remain episomal or integrate into the host cell genome. The vector can have the ability to self-replicate or not (i.e. drive only transient expression in a cell). The term includes recombinant expression cassettes that contain only the minimum elements needed for transcription of the recombinant nucleic acid. The vector of the invention can a “recombinant vector” which is by definition a man-made vector. The vector can also be a viral vector including lentiviral, retroviral, adenoviral and adeno-associated viral vectors.

“Reduction” or “reducing” or “lower” as used herein refers to a statistically significant reduction, more particularly said statistically significant reduction is an at least 10%, 15%, 20%, 25%, 30%, 35%, 40%, 45% or 50% reduction compared to the control situation.

“Increasing” or “increase” or “enhancing” or “promoting” or “stimulating” as used herein interchangeable and refer to a statistically significant increase, more particularly said statistically significant increase is an at least 10%, 15%, 20%, 25%, 30%, 35%, 40%, 45% or 50% increase compared to the control situation.

The term “statistically significantly” different is well known by the person skilled in the art. Statistical significance plays a pivotal role in statistical hypothesis testing. It is used to determine whether the null hypothesis should be rejected or retained. The null hypothesis is the default assumption that nothing happened or changed, hence that there is no difference for example in the recombination efficiency of a particular LoxPsym site compared to the recombination efficiency of the standard LoxPsym site as depicted in SEQ ID No. 128. For the null hypothesis to be rejected, an observed result has to be statistically significant, i.e. the observed p-value is less than the pre-specified significance level a. The p-value of a result, p, is the probability of obtaining a result at least as extreme, given that the null hypothesis were true. In one embodiment, a is 0.05. In a more particular embodiment, a is 0.01. In an even more particular embodiment, a is 0.001.

Yeasts are eukaryotic, single-celled microorganisms classified as members of the fungus kingdom and like all fungi, yeast may have asexual and sexual reproductive cycles. The most common mode of vegetative growth in yeast is asexual reproduction by budding. Here, a small bud or daughter cell, is formed on the parent cell. The nucleus of the parent cell splits into a daughter nucleus and migrates into the daughter cell. The bud continues to grow until it separates from the parent cell, forming a new cell. This reproduction cycle is independent of the yeast's ploidy, thus both haploid and diploid yeast cells can duplicate as described above. Haploid cells have in general a lower fitness and they often die under high-stress conditions such as nutrient starvation, while under the same conditions, diploid cells can undergo sporulation, entering sexual reproduction (meiosis) and producing a variety of haploid spores or haploid segregants, which can go on to mate (conjugate), reforming the diploid. Haploid cells contain one set of chromosomes, while diploid cells contain two. A haploid segregant as used herein is equivalent as a haploid spore, the result of sporulation.

The budding yeast Saccharomyces cerevisiae reproduces by mitosis as diploid cells when nutrients are abundant, but when starved, this yeast undergoes meiosis to form haploid spores. Haploid cells may then reproduce asexually by mitosis.

“Engineering” or “engineered” as used herein refers to genetic engineering, a technique whereby an organism's genome is modified using biotechnology. This includes but is not limited to the transfer of genes within and across species boundaries, deleting fragments of genes or deleting whole genes, modifying the DNA sequence of an organism by deleting, inserting or substituting one or more nucleic acid molecules. Means and methods to engineer microorganisms, particularly yeasts are well known by the person skilled in the art. The most known techniques involve traditional genetic transformation of yeast and recombinant DNA techniques. Nowadays, the most attractive technique to engineer a microorganism is by the use of nucleases, such as zinc-finger nucleases (ZFNs), Transcription Activator-Like Effector Nucleases (TALENs), meganucleases but especially the CRISPR-Cas system as described earlier.

### Cre/LoxP Recombination as Valuable Genetic Engineering Tool

DNA recombination is a process by which pieces of DNA are broken and recombined to produce new combinations of alleles. Being fundamental in creating genetic diversity in all organisms, site specific recombination has also proven to be a powerful tool in genetic engineering, systems biology and studies on developmental biology over the past decades.

Cre/LoxP is a widely used site-specific DNA recombination system derived from bacteriophage P1. Cre recombinase catalyzes a site-specific recombination reaction between two LoxP sites and does not require accessory factors (Guo et al Nature 389:40-46). The LoxP site is 34 base pairs (bp) in length, consisting of two 13 bp inverted repeats separated by an 8 bp asymmetric spacer sequence. The Cre/LoxP system can be used to generate deletions, inversions, insertions (transpositions), or translocations depending on the orientation and location of LoxP sites specified in a given system (Nagy 2000 Genesis 26:99). The simplicity of the Cre/LoxP system has led to its use in both in vivo and in vitro applications. Previous in vivo applications include targeted gene knock-out, gene replacement and more (Zou et al 1994 Curr Biol 4:1099-1103; Lewandoski & Martin 1997 Nat Genet 17:223-225) and in vitro applications comprise high-throughput DNA cloning and adenoviral vector construction (Marsischky & LaBaer 2004 Genome Res 14:2020-2028; Parks et al 1999 Gene Ther 10:2667-2672). The general goal of most existing Cre/LoxP applications is to recover a single recombination event at defined positions. If LoxP sites encode a symmetric spacer region (LoxPsym), rearrangements are orientation-independent and DNA fragments between two LoxPsym sites should undergo deletions or inversions with equal frequency (Hoess et al 1986 Nucleic Acids Res 14:2287-2300; Shen et al 2016 Genome Res 26:36-49).

LoxPsym sites comprise a left and right end, i.e. “LE” Cre recognition site, or “arm”, a right end, i.e. “RE” Cre recognition site, or “arm”, and sandwich between the LE and RE arms, i.e. a spacer region. In most wild type and mutant LoxP sites, the LE and RE arms are each 13 basepairs (bp) in length. In LoxPsym sites the LE and RE arms are inverted repeats. A non-limiting example of a LE sequence is 5′-ATAACTTCGTATA-3′ and of a RE sequence is 5′-TATACGAAGTTAT-3′. The spacer region is 8 bp in length. Each base in the spacer region is conventionally named 1, 2, 3, 4, 5, 6, 7, or 8, according to its order (5′->3′) in the sequence. Cre-LoxP sites mediate site specific intra- or inter-strand exchange of DNA molecules catalyzed by Cre recombinase.

### Novel LoxPsym Sites

The efficiency of Cre-Lox recombination events is a major determinant in setting up a genetic engineering exercise. Therefore, the inventors of current application developed 63 variants of the standard LoxPsym site with variable recombination efficiencies. Interestingly, 36 new LoxPsym sites appear more efficient than the originally described LoxPsym site, reaching nearly double as high recombination efficiencies, while 27 sites showed a reduced recombination efficiency compared to the standard LoxPsym site. Both groups have their value as genetic engineering tools as in particular cases a high or low recombination efficiency can be desired.

In a first aspect, the application provides a LoxPsym site or alternatively phrased a LoxPsym sequence or LoxPsym oligonucleotide, having the following formula:
5′-ATAACTTCGTATA—spacer—TATACGAAGTTAT-3′, wherein the spacer is selected from the list consisting of SEQ ID No. 1-63. The LoxPsym site has a nucleotide sequence derived from a wild-type E. coli P1 phage IoxP site. In one embodiment, the LoxPsym site is cleaved in the presence of recombinase Cre.

“Cre recombinase” or “recombinase Cre” or “Cre” as used herein refers to a tyrosine recombinase enzyme derived from the P1 bacteriophage (Uniprot ID: Q71TG5) and its amino acid sequence is depicted in SEQ ID No. 129.

(Cre recombinase)
SEQ ID No. 129
MSNLLTVHQNLPALPVDATSDEVRKNLMDMFRDRQAFSEHTWKMLLSVC
RSWAAWCKLNNRKWFPAEPEDVRDYLLYLQARGLAVKTIQQHLGQLNML
HRRSGLPRPSDSNAVSLVMRRIRKENVDAGERAKQALAFERTDFDQVRS
LMENSDRCQDIRNLAFLGIAYNTLLRIAEIARIRVKDISRTDGGRMLIH
IGRTKTLVSTAGVEKALSLGVTKLVERWISVSGVADDPNNYLFCRVRKN
GVAAPSATSQLSTRALEGIFEATHRLIYGAKDDSGQRYLAWSGHSARVG
AARDMARAGVSIPEIMQAGGWTNVNIVMNYIRNLDSETGAMVRLLEDGD

The enzyme uses a topoisomerase I-like mechanism to carry out site specific recombination events. The enzyme (38 kDa) is a member of the integrase family of site-specific recombinases and it is known to catalyse the site specific recombination event between two DNA recognition sites (LoxP or LoxPsym sites). This 34 nucleotide long LoxP recognition site consists of two palindromic sequences of 13 nucleotides which flank an 8 nucleotides short spacer region. The products of Cre-mediated recombination at LoxP sites are dependent upon the location and relative orientation of the LoxP sites. Two separate DNA species both containing LoxP sites can undergo fusion as the result of Cre mediated recombination. DNA sequences found between two LoxP sites are said to be “floxed”. In this case the products of Cre mediated recombination depends upon the orientation of the LoxP sites. DNA found between two LoxP sites oriented in the same direction will be excised as a circular loop of DNA whilst intervening DNA between two LoxP sites that are opposingly orientated will be inverted (Nagy 2000 Genesis 26:99-109). The enzyme requires no additional cofactors (such as ATP) or accessory proteins for its function (Abremski and Hoess 1984 J Biol Chem 259:1509-1514). If LoxP sites encode a symmetric spacer region (LoxPsym), rearrangements are orientation-independent and DNA fragments between two LoxPsym sites undergo deletions or inversions with equal frequency (Hoess et al 1986 Nucleic Acids Res 14:2287-2300; Shen et al 2016 Genome Res 26:36-49).

TABLE 1
Overview of the novel LoxPsym sites and the spacers
thereof and the recombination efficiency.

| SEQ | | SEQ | | Recombination |
|---|---|---|---|---|
| ID No. | spacer | ID No. | LoxPsym | efficiency (%) |
| 1 | GAGTACTC | 65 | ATAACTTCGTATAGAGTACTCTATACGAAGTTAT | 13.4 |
| 2 | AGCTAGCT | 66 | ATAACTTCGTATAAGCTAGCTTATACGAAGTTAT | 32.6 |
| 3 | CTATATAG | 67 | ATAACTTCGTATACTATATAGTATACGAAGTTAT | 34.1 |
| 4 | CTTTAAAG | 68 | ATAACTTCGTATACTTTAAAGTATACGAAGTTAT | 34.8 |
| 5 | AGATATCT | 69 | ATAACTTCGTATAAGATATCTTATACGAAGTTAT | 35.1 |
| 6 | TTATATAA | 70 | ATAACTTCGTATATTATATAATATACGAAGTTAT | 35.8 |
| 7 | GATTAATC | 71 | ATAACTTCGTATAGATTAATCTATACGAAGTTAT | 36.9 |
| 8 | GGATATCC | 72 | ATAACTTCGTATAGGATATCCTATACGAAGTTAT | 37.1 |
| 9 | GCTTAAGC | 73 | ATAACTTCGTATAGCTTAAGCTATACGAAGTTAT | 37.2 |
| 10 | GGTTAACC | 74 | ATAACTTCGTATAGGTTAACCTATACGAAGTTAT | 38.0 |
| 11 | CTCTAGAG | 75 | ATAACTTCGTATACTCTAGAGTATACGAAGTTAT | 38.2 |
| 12 | ACATATGT | 76 | ATAACTTCGTATAACATATGTTATACGAAGTTAT | 38.3 |
| 13 | TCATATGA | 77 | ATAACTTCGTATATCATATGATATACGAAGTTAT | 39.9 |
| 14 | GTGTACAC | 78 | ATAACTTCGTATAGTGTACACTATACGAAGTTAT | 40.3 |
| 15 | ACCTAGGT | 79 | ATAACTTCGTATAACCTAGGTTATACGAAGTTAT | 41.4 |
| 16 | ATTTAAAT | 80 | ATAACTTCGTATAATTTAAATTATACGAAGTTAT | 42.2 |
| 17 | ATATATAT | 81 | ATAACTTCGTATAATATATATTATACGAAGTTAT | 43.1 |
| 18 | TCTTAAGA | 82 | ATAACTTCGTATATCTTAAGATATACGAAGTTAT | 43.3 |
| 19 | AGTTAACT | 83 | ATAACTTCGTATAAGTTAACTTATACGAAGTTAT | 43.4 |
| 20 | GGCTAGCC | 84 | ATAACTTCGTATAGGCTAGCCTATACGAAGTTAT | 43.6 |
| 21 | AAGTACTT | 85 | ATAACTTCGTATAAAGTACTTTATACGAAGTTAT | 44.1 |
| 22 | GTCTAGAC | 86 | ATAACTTCGTATAGTCTAGACTATACGAAGTTAT | 44.3 |
| 23 | TTTTAAAA | 87 | ATAACTTCGTATATTTTAAAATATACGAAGTTAT | 44.5 |
| 24 | GGGTACCC | 88 | ATAACTTCGTATAGGGTACCCTATACGAAGTTAT | 45.6 |
| 25 | AACTAGTT | 89 | ATAACTTCGTATAAACTAGTTTATACGAAGTTAT | 46.0 |
| 26 | GAATATTC | 90 | ATAACTTCGTATAGAATATTCTATACGAAGTTAT | 46.2 |
| 27 | CGTTAACG | 91 | ATAACTTCGTATACGTTAACGTATACGAAGTTAT | 46.9 |
| 28 | AAATATTT | 92 | ATAACTTCGTATAAAATATTTTATACGAAGTTAT | 47.5 |
| 29 | AGGTACCT | 93 | ATAACTTCGTATAAGGTACCTTATACGAAGTTAT | 49.1 |
| 30 | ACGTACGT | 94 | ATAACTTCGTATAACGTACGTTATACGAAGTTAT | 51.1 |
| 31 | CCTTAAGG | 95 | ATAACTTCGTATACCTTAAGGTATACGAAGTTAT | 51.2 |
| 32 | CTGTACAG | 96 | ATAACTTCGTATACTGTACAGTATACGAAGTTAT | 53.1 |
| 33 | CCATATGG | 97 | ATAACTTCGTATACCATATGGTATACGAAGTTAT | 53.9 |
| 34 | CGATATCG | 98 | ATAACTTCGTATACGATATCGTATACGAAGTTAT | 54.4 |
| 35 | ATCTAGAT | 99 | ATAACTTCGTATAATCTAGATTATACGAAGTTAT | 56.5 |
| 36 | CACTAGTG | 100 | ATAACTTCGTATACACTAGTGTATACGAAGTTAT | 57.5 |
| 37 | TGATATCA | 101 | ATAACTTCGTATATGATATCATATACGAAGTTAT | 58.8 |
| 38 | TCGTACGA | 102 | ATAACTTCGTATATCGTACGATATACGAAGTTAT | 59.0 |
| 39 | GCATATGC | 103 | ATAACTTCGTATAGCATATGCTATACGAAGTTAT | 59.1 |
| 40 | CAATATTG | 104 | ATAACTTCGTATACAATATTGTATACGAAGTTAT | 59.7 |
| 41 | TTCTAGAA | 105 | ATAACTTCGTATATTCTAGAATATACGAAGTTAT | 62.3 |
| 42 | GTATATAC | 106 | ATAACTTCGTATAGTATATACTATACGAAGTTAT | 64.3 |
| 43 | GTTTAAAC | 107 | ATAACTTCGTATAGTTTAAACTATACGAAGTTAT | 64.6 |
| 44 | GCCTAGGC | 108 | ATAACTTCGTATAGCCTAGGCTATACGAAGTTAT | 65.1 |
| 45 | TAATATTA | 109 | ATAACTTCGTATATAATATTATATACGAAGTTAT | 65.5 |
| 46 | CCCTAGGG | 110 | ATAACTTCGTATACCCTAGGGTATACGAAGTTAT | 67.2 |
| 47 | GACTAGTC | 111 | ATAACTTCGTATAGACTAGTCTATACGAAGTTAT | 67.4 |
| 48 | TGGTACCA | 112 | ATAACTTCGTATATGGTACCATATACGAAGTTAT | 69.1 |
| 49 | CGCTAGCG | 113 | ATAACTTCGTATACGCTAGCGTATACGAAGTTAT | 69.4 |
| 50 | TAGTACTA | 114 | ATAACTTCGTATATAGTACTATATACGAAGTTAT | 70.3 |
| 51 | ACTTAAGT | 115 | ATAACTTCGTATAACTTAAGTTATACGAAGTTAT | 70.5 |
| 52 | AATTAATT | 116 | ATAACTTCGTATAAATTAATTTATACGAAGTTAT | 71.1 |
| 53 | TGCTAGCA | 117 | ATAACTTCGTATATGCTAGCATATACGAAGTTAT | 72.0 |
| 54 | TACTAGTA | 118 | ATAACTTCGTATATACTAGTATATACGAAGTTAT | 75.3 |
| 55 | CCGTACGG | 119 | ATAACTTCGTATACCGTACGGTATACGAAGTTAT | 76.0 |
| 56 | CATTAATG | 120 | ATAACTTCGTATACATTAATGTATACGAAGTTAT | 79.1 |
| 57 | GCGTACGC | 121 | ATAACTTCGTATAGCGTACGCTATACGAAGTTAT | 79.2 |
| 58 | CAGTACTG | 122 | ATAACTTCGTATACAGTACTGTATACGAAGTTAT | 79.9 |
| 59 | TCCTAGGA | 123 | ATAACTTCGTATATCCTAGGATATACGAAGTTAT | 80.0 |
| 60 | TTGTACAA | 124 | ATAACTTCGTATATTGTACAATATACGAAGTTAT | 80.6 |
| 61 | CGGTACCG | 125 | ATAACTTCGTATACGGTACCGTATACGAAGTTAT | 81.5 |
| 62 | TGTTAACA | 126 | ATAACTTCGTATATGTTAACATATACGAAGTTAT | 82.4 |
| 63 | TATTAATA | 127 | ATAACTTCGTATATATTAATATATACGAAGTTAT | 89.3 |

In one embodiment, the LoxPsym site has a recombination efficiency that is lower than the recombination efficiency of the standard LoxPsym site described by Hoess et al 1986, more particularly the LoxPsym site as depicted in SEQ ID No. 128 (ATAACTTCGTATAATGTACATTATACGAAGTTAT) and comprising the spacer as depicted in SEQ ID No. 64 (ATGTACAT). The recombination efficiency of SEQ ID No. 128 is 47.0%.

Hence, a LoxPsym site is provided having the following formula:
5′-ATAACTTCGTATA—spacer—TATACGAAGTTAT-3′, wherein the spacer is selected from the list consisting of SEQ ID No. 1-27 or wherein the LoxPsym site is selected from the list consisting of SEQ ID No. 65-91. In a particular embodiment, the LoxPsym site has a recombination efficiency that is at least 10% lower compared to the recombination efficiency of the SEQ ID No. 128, more particularly the LoxPsym site comprises a spacer selected from the list consisting of SEQ ID No. 1-16. In a more particular embodiment, the LoxPsym site has a recombination efficiency that is at least 15% lower compared to the recombination efficiency of the SEQ ID No. 128, more particularly the LoxPsym site comprises a spacer selected from the list consisting of SEQ ID No. 1-12. In an even more particular embodiment, the LoxPsym site has a recombination efficiency that is at least 20% lower compared to the recombination efficiency of the SEQ ID No. 128, more particularly the LoxPsym site comprises a spacer selected from the list consisting of SEQ ID No. 1-9.

In another embodiment, the LoxPsym site has a recombination efficiency that is higher than the recombination efficiency of the standard LoxPsym site described by Hoess et al 1986, more particularly the LoxPsym site as depicted in SEQ ID No. 128 and having the spacer as depicted in SEQ ID No. 64. Hence, a LoxPsym site is provided having the following formula:
5′-ATAACTTCGTATA—spacer—TATACGAAGTTAT-3′, wherein the spacer is selected from the list consisting of SEQ ID No. 28-63 or wherein the LoxPsym site is selected from the list consisting of SEQ ID No. 92-127. In a particular embodiment, the LoxPsym site has a recombination efficiency that is at least 10% higher compared to the recombination efficiency of the SEQ ID No. 128, more particularly the LoxPsym site comprises a spacer selected from the list consisting of SEQ ID No. 32-63. In a more particular embodiment, the LoxPsym site has a recombination efficiency that is at least 15% higher compared to the recombination efficiency of the SEQ ID No. 128, more particularly the LoxPsym site comprises a spacer selected from the list consisting of SEQ ID No. 34-63. In an even more particular embodiment, the LoxPsym site has a recombination efficiency that is at least 20% lower compared to the recombination efficiency of the SEQ ID No. 128, more particularly the LoxPsym site comprises a spacer selected from the list consisting of SEQ ID No. 35-63.

In third aspect, a vector is provided comprising any of the herein disclosed LoxPsym sites. In a particular embodiment, said vector comprise a nucleic acid sequence flanked at both the 5′ and 3′ end by a LoxPsym site. In a particular embodiment, the nucleic acid sequence is flanked by the LoxPsym site. In another particular embodiment, the LoxPsym site that flank the nucleic acid sequence are different. In another particular embodiment, the nucleic acid sequence is a gene of interest or fragment thereof, a promoter or fragment thereof, a terminator or fragment thereof or any coding, non-coding or regulatory nucleic acid sequence (e.g. a 3′ end region involved in transcription termination or polyadenylation).

In yet another aspect, a host cell is provided comprising any of the vectors as described above or comprising any of the herein disclosed LoxPsym sites. In a particular embodiment, the host cell is a microorganism, a plant cell, an insect cell, a mammalian cell, or a yeast cell. In a particular embodiment, one or more LoxPsym sites according to any embodiment of the first aspect are amplified, for example, by means of PCR and the obtained product, preferably being a linear product, is transformed directly to the host cell. In another particular embodiment, one or more LoxPsym sites according to any embodiment of the first aspect is incorporated by means of the vector according to any embodiment of the third aspect. It should be understood that any method known to a person skilled in the art can be used to incorporate one or more LoxPsym sites into the host cell as disclosed herein, without departing from the scope of the present application. In an even more particular embodiment, the host cell is a bacterium such as Escherichia coli, or a yeast cell, even more particular a Saccharomyces, a Yarrowia or a Pichia yeast. In another particular embodiment said host cell is a cell of a species selected form a group consisting of Saccharomyces cerevisiae, Escherichia coli, Zea mays and Yarrowia lipolytica.

### Novel Orthogonal LoxPsym Sites

Microbial biotechnology or microbial engineering explores the power of bacteria and yeasts to obtain economically valuable products or activities at an industrial scale. Synthetic biology and recombinant DNA technologies has enabled the expression of heterologous pathways in host cells which are not restricted to microorganisms but extent to plant cells, insect cells and mammalian cells. When cells are used as factories, preferably whole biosynthetic pathways are inserted in the cell's genome. Since high expression of all biosynthesis genes is often not leading to maximal product yields (e.g. intermediates can accumulate that feedback on the system, are toxic or lead to suboptimal growth), the expression levels of several modules or genes should be optimized and adjusted to one another.

The current available approaches based for example on simply trying many different combinations, directed evolution, computational predictions, self-tuning systems with feedback inhibition, etc. are laborsome, expensive and take a lot of time.

The inventors of current application developed a simultaneous in vivo cloning and testing approach based on the well-known Cre-Lox system. Several of the 63 variants of the LoxPsym site described above were tested for cross-reaction. Specifically, 1056 interactions between LoxPsym variants were tested in a fluorescence-based assay to identify orthogonal LoxPsym variants. From this interaction-matrix as shown in FIG. 3G, it can be found that several sets of orthogonal LoxPsym sites can be selected, the largest sets including 16 variants that are all cleavable by Cre but that do not recombine among each other. An example of such a set is a set consisting of the LoxPsym sites selected from the list consisting of SEQ ID No. 65, 69-71, 74, 77, 82, 84, 100, 105, 107-109, 112, 121 and 124 or a set consisting of the LoxPsym sites comprising a spacer selected from the list consisting of SEQ ID No. 1, 5-7, 10, 13, 18, 20, 36, 41, 43-45, 48, 57 and 60. Other non-limiting examples of sets of orthogonal LoxPsym sites consist of the LoxPsym sites selected from the list consisting of SEQ ID No. 70, 77, 82, 101-102, 105, 107, 109, 112, 114, 117-118, 123-124, 126-127 or consisting of SEQ ID No. 65, 71-74, 84, 86, 88, 90, 103, 106-108, 111, 121, 124 or consisting of SEQ ID No. 72, 74, 77-78, 80, 82, 84, 86, 88, 102, 106, 109, 114, 118, 123, 127 or consisting of SEQ ID No. 65, 70-71, 73, 80, 90, 101, 103, 105, 108, 111-112, 117, 121, 124, 126. Also, smaller sets of LoxPsym sites are provided. A non-limiting example is a set consisting of LoxPsym sites selected from the list consisting of SEQ ID No. 69, 70, 74, 77, 84, 86, 88, 100, 108, 109, 114 and 124.

The herein disclosed orthogonal LoxPsym sites can for example be used as follows: a cell is transformed with one or more constructs comprising genes A, B, C, . . . . Upstream of the genes, several promoter elements are present being separated by LoxPsym sites, in such a way that all promoter elements in front of gene A are separated by the same LoxPsym site but for every other gene different LoxPsym sites are used (FIG. 1). The same approach can be taken for optimizing the terminator sequence downstream of the genes: several terminator elements are separated by LoxPsym sites, i.e. the same LoxPsym site for all terminator elements of one gene but different from the LoxPsym sites of the promoter elements of that gene and different from the LoxPsym sites of other genes (FIG. 1). By inducing the expression of Cre recombinase in the cells, different combinations are made between the promoters elements and terminators elements separately and for several genes simultaneously (orthogonal recombination). The genetic diversity of the resulting cell population results in phenotypic diversity of the trait of interest, such as the production of a high valuable-compound (FIG. 1). The best performing cells are then sequenced to reveal the optimized promoter/terminator combinations per gene and optionally re-engineered in a clean way.

In a second aspect, the invention provides orthogonal LoxPsym sites that may be used for assembling nucleic acid constructs. The term “orthogonal” or “orthogonality” in (synthetic) biology describes the inability of two or more molecules, similar in composition and/or function, to interact with one another or affect their respective substrates, hence “orthogonal” as used herein refers to “independently acting” or “non-cross reacting”.

LoxPsym sites are thus “orthogonal” when—in the presence of Cre recombinase—the LoxPsym sites can only recombine with LoxPsym sites having an identical nucleic acid sequence and are not recombining with LoxPsym sites having a different nucleic acid sequence. This is a huge advantage since specific recombination events can be initiated simultaneously without influencing each other. Orthogonal recombination is currently obtained by using multiple recombinases which recognize distinct recombination sites (Wang et al 2022 iScience 25:103716). However, the number of such non-cross-reactive recombinases remains limited and in cellulo expression can potentially have yield dragging effects. The developed orthogonal LoxPsym variants herein disclosed overcome these disadvantages.

The application provides a selection of orthogonal or non-cross reacting LoxPsym sites. More particularly, mutant LoxPsym sites or sequences that in the presence of recombinase Cre can be cleaved and wherein a specific DNA recombination can occur between the LoxPsym sites having identical nucleotide sequences but wherein no recombination can occur between LoxPsym sites having a different nucleotide sequence. In a particular embodiment, the LoxPsym sites comprise or consist of the formula: 5′-ATAACTTCGTATA—spacer—TATACGAAGTTAT-3′, wherein the spacer is selected from the list consisting of SEQ ID No. 1-63.

In one embodiment, orthogonal LoxPsym sites comprising or consisting of the formula:
5′-ATAACTTCGTATA—spacer—TATACGAAGTTAT-3′ are provided, wherein the spacer is selected from the list consisting of SEQ ID No. 1-63, and wherein the LoxPsym sites in the presence of recombinase Cre can be cleaved and wherein a specific DNA recombination can only occur between LoxPsym sites having identical nucleotide sequences or between LoxPsym sites comprising the same nucleotides on position 2-7 of the spacer. In a particular embodiment, recombination between said LoxPsym sites will not occur when said sites comprise a different nucleotide on position 2, 3, 4, 5, 6 and/or 7 of the spacer.

In a particular embodiment, a LoxPsym site, a mutant LoxPsym site or an orthogonal LoxPsym site is provided with the following properties:
comprising or consisting of a nucleic acid sequence of the following formula:

5′-ATAACTTCGTATA-spacer-TATACGAAGTTAT-3′,

wherein the spacer consists of 8 bases and selected from the list consisting of SEQ ID No. 1-63; and
when in the presence of recombinase Cre, the LoxPsym site can only recombine with a LoxPsym site having an identical nucleotide sequence or with a LoxPsym site comprising or consisting of the formula 5′-ATAACTTCGTATA—spacer—TATACGAAGTTAT-3′ wherein the spacer is selected from the list consisting of SEQ ID No. 1-63 and comprising an identical nucleotide sequence from position 2 until position 7 of the spacer.

“From position 2 until position 7 of the spacer” means that both position 2 and 7 are included. In the presence of Cre recombinase, the orthogonal LoxPsym site is not able to recombine with a LoxPsym site having a different nucleotide on position 2, 3, 4, 5, 6 or 7 of the spacer.

In another aspect, a set of LoxPsym sites is provided comprising at least a first and second LoxPsym site, the LoxPsym sites are selected from the list consisting of SEQ ID No. 65-127 or have the following formula: 5′-ATAACTTOGTATA—spacer—TATACGAAGTTAT-3′, wherein the spacer is selected from the list consisting of SEQ ID No. 1-63. In one embodiment, in the presence of recombinase Cre a specific DNA recombination between the first and second LoxPsym site can only occur when the first and second LoxPsym site have the same nucleic acid sequence or share the same nucleotides on positions 2-7 of the spacer. In another embodiment, in the presence of recombinase Cre a specific DNA recombination between any LoxPsym site from the set and any other LoxPsym site from the set can occur when said LoxPsym sites share the same nucleotides on positions 2-7 of the spacer. In a particular embodiment, said set of LoxPsym sites does not comprise SEQ ID No. 88 and 89, SEQ ID No. 88 and 81, SEQ ID No. 90 and 83 or SEQ ID No. 78 and 103.

In another embodiment, in the presence of recombinase Cre a specific DNA recombination between the first and second LoxPsym site of said set cannot occur when the spacers of the first and second LoxPsym site differ and/or when the spacer of the first LoxPsym site comprises a different nucleotide compared to the spacer of the second LoxPsym site on position 2, 3, 4, 5, 6 and/or 7, more particularly on position 2, 3, 6 and/or 7 of the spacer, except when the first and second LoxPsym sites comprise the spacer combinations GGGTACCC-AACTAGTT, GGGTACCC-ATATATAT, GAATATTC-AGTTAACT, GAGTACTC-AAATATTT or GTGTACAC-GCATATGC or alternatively phrased, except when the first and second LoxPsym sites comprise the spacers GGGTACCC and AACTAGTT, GGGTACCC and ATATATAT, GAATATTC and AGTTAACT, GAGTACTC and AAATATTT or GTGTACAC and GCATATGC respectively.

In another embodiment, in the presence of recombinase Cre a specific DNA recombination between the first and second LoxPsym site of said set cannot occur when the first and second LoxPsym sites comprise a different nucleotide on position 2, 3, 4, 5, 6 and/or 7 of the spacer, except when the first and second LoxPsym sites comprise the spacer combinations GGGTACCC-AACTAGTT, GGGTACCC-ATATATAT, GAATATTC-AGTTAACT, GAGTACTC-AAATATTT, or GTGTACAC-GCATATGC.

In another embodiment, a set of at least two LoxPsym sites is provided, the LoxPsym sites comprise or consist of the formula: 5′-ATAACTTCGTATA—spacer—TATACGAAGTTAT-3′, wherein the spacer is selected from the list consisting of SEQ ID No. 1-63, and wherein the at least two LoxPsym sites comprise a different nucleotide on position 2, 3, 4, 5, 6 and/or 7 of the spacer, and wherein the set does not comprise the LoxPsym sites depicted in SEQ ID No. 88 and 89, SEQ ID No. 88 and 81, SEQ ID No. 90 and 83, SEQ ID No. 65 and 92, or SEQ ID No. 78 and 103.

In one embodiment, a set of LoxPsym sites is provided comprising at least a first and second LoxPsym site, the LoxPsym sites having the following formula: 5′-ATAACTTCGTATA—spacer—TATACGAAGTTAT-3′, wherein the spacer is selected from the list consisting of SEQ ID No. 1-63, and
i) wherein in the presence of recombinase Cre a specific DNA recombination between the first and second LoxPsym site can occur when the first and second LoxPsym site have the same nucleic acid sequence or alternatively phrased when the first and second LoxPsym sites are identical; and
ii) wherein in the presence of recombinase Cre a specific DNA recombination between the first and second LoxPsym site cannot occur when the first and second LoxPsym sites comprise a different nucleotide on position 2, 3, 4, 5, 6 and/or 7 of the spacer, except for the spacer combinations GGGTACCC-AACTAGTT, GGGTACCC-ATATATAT, GAATATTC-AGTTAACT, GAGTACTC-AAATATTT or GTGTACAC-GCATATGC.

In another embodiment, a set of at least two LoxPsym sites is provided, the LoxPsym sites having the following formula: 5′-ATAACTTCGTATA—spacer—TATACGAAGTTAT-3′, wherein the spacer is selected from the list consisting of SEQ ID No. 1-63, wherein the at least two LoxPsym sites differ from each other in at least one nucleotide residue on position 2-7 of the spacer. In a particular embodiment, said at least two LoxPsym sites do not comprise the spacers GGGTACCC and AACTAGTT, GGGTACCC and ATATATAT, GAATATTC and AGTTAACT, GAGTACTC and AAATATTT or GTGTACAC and GCATATGC. In another particular embodiment, said at least two LoxPsym sites do not comprise SEQ ID No. 88 and 89, SEQ ID No. 88 and 81, SEQ ID No. 90 and 83, SEQ ID No. 65 and 92 or SEQ ID No. 78 and 103. In a particular embodiment, the set comprises at least 3, at least 4, at least 5, at least 6, at least 7, at least 8, at least 9, at least 10, at least 11, at least 12, at least 13, at least 14, at least 15 or at least 16 LoxPsym sites. In a more particular embodiment, the at least 2, 3, 4, 5, 6, 7, 8, 9, 10, 11, 12, 13, 14, 15 or 16 LoxPsym sites are selected from the list consisting of SEQ ID No. 65, 69-71, 74, 77, 82, 84, 100, 105, 107-109, 112, 121 and/or 124.

In another aspect, a chimeric gene construct is provided comprising one or more LoxPsym sites herein described. In one embodiment, the chimeric gene construct comprising at least two LoxPsym sites. In a particular embodiment, the chimeric gene construct comprises a gene of interest or fragment thereof, a promoter or fragment thereof, a terminator or fragments thereof of any other coding, non-coding or regulatory nucleic acid sequence, flanked at the 5′ and/or at 3′ end by one or more of the LoxPsym sites herein disclosed.

In another aspect, a vector is provided comprising one or more LoxPsym sites herein described or comprising any of the above described chimeric gene constructs.

Also a host cell is provided comprising said vector or comprising any of the LoxPsym sites herein disclosed. In particular embodiments, the host cell is a microorganism, a plant cell, an insect cell or a mammalian cell. In particular embodiments, the host cell is a bacterial or yeast cell. In more particular embodiments, the host cell is a yeast, even more particularly Saccharomyces, Yarrowia or Pichia yeast, most particularly S. cerevisiae. In another particular embodiment said host cell is a cell of a species selected form a group consisting of Saccharomyces cerevisiae, Escherichia coli, Zea mays and Yarrowia lipolytica.

In another embodiment, a set or combination or selection of vectors is provided wherein each vector comprises a nucleic acid sequence flanked by one of the LoxPsym sites herein disclosed at the 5′ end of said nucleic acid sequence and by the same LoxPsym site at the 3′ end of said nucleic acid sequence, wherein every vector of said set of vectors comprises a different LoxPsym site. Said nucleic acid sequence can be a promoter or promoter element, terminator or terminator element, an exon or exon fragment, an intron or intron fragment, or any other regulatory, coding or non-coding DNA sequence.

Methods of In Vivo and/or In Vitro Recombination

As described herein, the LoxPsym sites according to the invention can be used for site-specific recombination events of one or more nucleic acid sequences in combination with the Cre recombinase.

In another aspect, the use is provided of any of the LoxPsym sites herein disclosed for in vivo and/or in vitro cloning.

More particularly, an in vivo or in vitro method is provided for obtaining a recombinant nucleic acid sequence or molecule comprising:
combining a nucleic acid sequence or molecule comprising at least two or more nucleic acid segments or elements and one or more site-specific recombinase recognition site that can be recognized by a recombinase, with a recombinase that recognizes the site-specific recombinase recognition site, such that the nucleic acid molecule or sequence is recombined to provide a recombinant nucleic acid molecule; and
optionally determining the sequence and/or determining the expression of the recombinant nucleic acid molecule or sequence subsequent to introducing the recombinant nucleic acid molecule or sequence in a cell.

In one embodiment, the site-specific recombinase recognition site or sites are LoxPsym sites and the recombinase is the Cre recombinase that recognizes the LoxPsym sites. In a particular embodiment, the LoxPsym sites are selected for the LoxPsym site herein disclosed, more particularly LoxPsym sites comprising or consisting of SEQ ID No. 65-127. In another particular embodiment, the DNA segments can be a nucleic acid sequence that encodes a protein or a non-coding RNA, promoter elements, terminator elements, or any other regulatory nucleic acid sequence.

Also provided is a method of replacing a DNA element A by a different DNA element B, the method comprising the steps of:
providing a nucleic acid molecule A′ comprising a LoxP site, DNA element A and a LoxP site in this 5′-3′ order and a nucleic acid molecule B′ comprising a LoxP site, DNA element B and a LoxP site in this 5′-3′ order, wherein at least one LoxP site is a LoxPsym site according to the invention, more particularly a LoxPsym site comprising a nucleic acid sequence as depicted in SEQ ID No. 65-127;
reacting nucleic acid molecule A′ and B′ in the presence of recombinase Cre, to obtain a nucleic acid molecule in which DNA element A is replaced by DNA element B.

In one embodiment, at least two, at least three, at least four or all LoxP sites are selected from the LoxPsym sites herein disclosed. In a particular embodiment, said LoxPsym sites are selected from the list consisting of SEQ ID No. 65-91, SEQ ID No. 65-80, SEQ ID No. 65-76, SEQ ID No. 65-73, SEQ ID No. 92-127, SEQ ID No. 96-127, SEQ ID No. 98-127, SEQ ID No. 99-127 and/or selected from the list consisting of SEQ ID No. 65, 69-71, 74, 77, 82, 84, 100, 105, 107-109, 112, 121 and 124.

### Method of Optimizing Gene Expression

A specific use of the orthogonal LoxPsym sites herein disclosed is the simultaneous in vivo cloning and phenotyping. Preferably, said in vivo cloning and phenotyping is performed in the same cell. More particularly, a method is provided of optimizing gene expression of one or more genes in a cell, comprising the steps of:
a. expressing or introducing in a cell, one or more vectors comprising one or more genes of interest under control of a promoter, the promoter comprising two or more promoter elements, wherein the two or more promoter elements are individually flanked or each flanked by an orthogonal LoxPsym site, wherein a different LoxPsym site is used per gene or per promoter;
b. optionally, the one or more vectors comprise a terminator sequence downstream of the one or more genes of interest, the terminator sequence comprising two or more terminator elements, wherein the two or more terminator elements are individually flanked or each flanked by an orthogonal LoxPsym site, wherein a different LoxPsym site is used per gene and wherein any of the LoxPsym sites from step b) are different to any of the LoxPsym sites used in step a);
c. expressing in said cell a recombinase Cre;
d. analysing the gene expression level of said gene of interest or analysing the phenotype of the cell.

Also provided is a method of optimizing gene expression of one or more genes in a cell, comprising the steps of:
a. expressing or introducing in a cell, one or more vectors comprising one or more genes of interest under control of a promoter, the one or more vectors comprise a terminator sequence downstream of the one or more genes of interest, the terminator sequence comprising two or more terminator elements, wherein the two or more terminator elements are individually flanked or each flanked by an orthogonal LoxPsym site, wherein a different LoxPsym site is used per gene or per terminator;
b. optionally, the promoter comprising two or more promoter elements, wherein the two or more promoter elements are individually flanked or each flanked by an orthogonal LoxPsym site, wherein a different LoxPsym site is used per gene and wherein any of the LoxPsym sites from step b) are different to any of the LoxPsym sites used in step a);
c. expressing in said cell a recombinase Cre;
d. analysing the gene expression level of said gene of interest or analysing the phenotype of the cell.

In one embodiment, the orthogonal LoxPsym site is selected from any of the LoxPsym sites herein disclosed, more particularly a LoxPsym site having the formula:
5′-ATAACTTCGTATA—spacer—TATACGAAGTTAT-3′,
wherein the spacer is selected from the list consisting of SEQ ID No. 1-63, and wherein in the presence of recombinase Cre a specific DNA recombination between the LoxPsym site and another LoxPsym site having the following formula: 5′-ATAACTTCGTATA—spacer—TATACGAAGTTAT-3′ and comprising a spacer selected from the list consisting of SEQ ID No. 1-63 cannot occur when the LoxPsym sites comprise a different nucleotide on position 2, 3, 6 and/or 7 of the spacer, except when the LoxPsym sites comprise the spacer combinations GGGTACCC-AACTAGTT, GGGTACCC-ATATATAT, GAATATTC-AGTTAACT, GAATATTC-AGTTAACT or GTGTACAC-GCATATGC.

In another embodiment, the orthogonal LoxPsym site is a LoxPsym site having the following formula:
5′-ATAACTTCGTATA—spacer—TATACGAAGTTAT-3′, wherein the spacer is selected from the list consisting of SEQ ID No. 1-63, and wherein in the presence of recombinase Cre a specific DNA recombination between the LoxPsym site and another LoxPsym site having the following formula: 5′-ATAACTTCGTATA—spacer—TATACGAAGTTAT and comprising a spacer selected from the list consisting of SEQ ID No. 1-63 can occur only when the spacers from both LoxPsym sites comprise the same nucleotide sequence between position 2 and 7 of the spacer and cannot occur when the LoxPsym sites comprise a different nucleotide on position 2, 3, 6 and/or 7 of the spacer, except for the spacer combinations GGGTACCC-AACTAGTT, GGGTACCC-ATATATAT, GAATATTC-AGTTAACT, GTGTACAC-GCATATGC.

In another embodiment, the orthogonal LoxPsym site is selected from any of the sets of LoxPsym sites herein disclosed, more particularly selected for a set of LoxPsym sites comprising at least two LoxPsym sites having the following formula: 5′-ATAACTTCGTATA—spacer—TATACGAAGTTAT-3′, wherein the spacer is selected from the list consisting of SEQ ID No. 1-63, and wherein the at least two LoxPsym sites comprise a different nucleotide on position 2, 3, 6 and/or 7 of the spacer, and wherein the set does not comprise the LoxPsym sites depicted in SEQ ID No. 88 and 89, SEQ ID No. 88 and 81, SEQ ID No. 90 and 83, SEQ ID No. 65 and 92, or SEQ ID No. 78 and 103.

In a particular embodiment, the orthogonal LoxPsym site is selected from SEQ ID No. 65, 69-71, 74, 77, 82, 84, 100, 105, 107-109, 112, 121 and/or 124 or selected from the list consisting of SEQ ID No. 65, 69-71, 74, 77, 82, 84, 100, 105, 107-109, 112, 121 and 124.

In one embodiment, the method further provides a step of sequencing or determining the sequence of the cell, more particularly to determine the sequence of the recombined nucleic acid molecule responsible for the phenotype.

“Phenotype” as used herein includes but is not limited to cell growth, reproductive fitness, synthesis of one or more compounds, detectable markers, or any other observable characteristic.

“Individually flanked” or “each flanked” as used herein means that a nucleic acid (e.g. a promoter or terminator element) comprises both at the S′ and at the 3′ end an additional element, such as a LoxPsym site.

Also a method of shuffling DNA elements is provided comprising the steps of:
providing a nucleic acid molecule comprising at least two DNA elements individually flanked by an orthogonal LoxP site;
reacting the nucleic acid molecule with recombinase Cre, to obtain a nucleic acid molecule in which the at least two DNA elements are reshuffled.

In one embodiment, the nucleic acid molecule is a gene promoter and the at least two DNA elements are promoter elements. In another embodiment, the nucleic acid molecule is a terminator sequence and the at least two DNA elements are terminator elements. In yet another embodiment, the nucleic acid molecule is a protein coding or non-coding gene and the at least two DNA elements are introns and/or exons.

In another embodiment, the orthogonal LoxP site is selected from any of the LoxPsym sites herein disclosed, more particularly a LoxPsym site having the formula:
5′-ATAACTTCGTATA—spacer—TATACGAAGTTAT,
wherein the spacer is selected from the list consisting of SEQ ID No. 1-63, and wherein in the presence of recombinase Cre a specific DNA recombination between the LoxPsym site and another LoxPsym site having the following formula: 5′-ATAACTTCGTATA—spacer—TATACGAAGTTAT and comprising a spacer selected from the list consisting of SEQ ID No. 1-63 cannot occur when the LoxPsym sites comprise a different nucleotide on position 2, 3, 6 and/or 7 of the spacer, except when the LoxPsym sites comprise the spacer combinations GGGTACCC and AACTAGTT, GGGTACCC and ATATATAT, GAATATTC and AGTTAACT, GAGTACTC and AAATATTT and/or GTGTACAC and GCATATGC.

In another embodiment, the orthogonal LoxPsym site is a LoxPsym site having the following formula:
5′-ATAACTTCGTATA—spacer—TATACGAAGTTAT, wherein the spacer is selected from the list consisting of SEQ ID No. 1-63, and wherein in the presence of recombinase Cre a specific DNA recombination between the LoxPsym site and another LoxPsym site having the following formula: 5′-ATAACTTOGTATA—spacer—TATACGAAGTTAT and comprising a spacer selected from the list consisting of SEQ ID No. 1-63 can occur only when the spacers from both LoxPsym sites comprise the same nucleotide sequence between position 2 and 7 of the spacer and cannot occur when the LoxPsym sites comprise a different nucleotide on position 2, 3, 6 and/or 7 of the spacer, except for the spacer combinations GGGTACCC and AACTAGTT, GGGTACCC and ATATATAT, GAATATTC and AGTTAACT, GAGTACTC and AAATATTT, and/or GTGTACAC and GCATATGC.

In another embodiment, the orthogonal LoxPsym site is selected from any of the sets of LoxPsym sites herein disclosed, more particularly selected for a set of LoxPsym sites comprising at least two LoxPsym sites having the following formula: S′-ATAACTTCGTATA—spacer—TATACGAAGTTAT-3′, wherein the spacer is selected from the list consisting of SEQ ID No. 1-63, and wherein the at least two LoxPsym sites comprise a different nucleotide on position 2, 3, 6 and/or 7 of the spacer, and wherein the set does not comprise the LoxPsym sites depicted in SEQ ID No. 88 and 89, SEQ ID No. 88 and 81, SEQ ID No. 90 and 83, SEQ ID No. 65 and 92, or SEQ ID No. 78 and 103.

In a particular embodiment, the orthogonal LoxPsym site is selected from SEQ ID No. 65, 69-71, 74, 77, 82, 84, 100, 105, 107-109, 112, 121 and/or 124 or selected from the list consisting of SEQ ID No. 65, 69-71, 74, 77, 82, 84, 100, 105, 107-109, 112, 121 and 124.

Also a method of modifying the expression of a gene is provided, comprising the steps of:
providing a nucleic acid molecule comprising the gene operably fused to a promoter, the promoter comprising two or more promoter elements individually flanked by an orthogonal LoxPsym site;
optionally, the nucleic acid molecule comprises a terminator sequence downstream of the gene, wherein the terminal sequence comprises two or more terminator elements individually flanked by an orthogonal LoxPsym site;
reshuffling the promoter elements and optionally the terminator elements by reacting the nucleic acid molecule with recombinase Cre.

In one embodiment, the orthogonal LoxPsym site is selected from any of the LoxPsym sites herein disclosed, more particularly a LoxPsym site having the formula:
5′-ATAACTTCGTATA—spacer—TATACGAAGTTAT-3′, wherein the spacer is selected from the list consisting of SEQ ID No. 1-63, and wherein in the presence of recombinase Cre a specific DNA recombination between the LoxPsym site and another LoxPsym site having the following formula: 5′-ATAACTTCGTATA—spacer—TATACGAAGTTAT-3′ and comprising a spacer selected from the list consisting of SEQ ID No. 1-63 cannot occur when the LoxPsym sites comprise a different nucleotide on position 2, 3, 6 and/or 7 of the spacer, except when the LoxPsym sites comprise the spacer combinations GGGTACCC-AACTAGTT, GGGTACCC-ATATATAT, GAATATTC-AGTTAACT, GAATATTC-AGTTAACT or GTGTACAC-GCATATGC.

In another embodiment, the orthogonal LoxPsym site is a LoxPsym site having the following formula:
5′-ATAACTTCGTATA—spacer—TATACGAAGTTAT-3′, wherein the spacer is selected from the list consisting of SEQ ID No. 1-63, and wherein in the presence of recombinase Cre a specific DNA recombination between the LoxPsym site and another LoxPsym site having the following formula: 5′-ATAACTTCGTATA—spacer—TATACGAAGTTAT and comprising a spacer selected from the list consisting of SEQ ID No. 1-63 can occur only when the spacers from both LoxPsym sites comprise the same nucleotide sequence between position 2 and 7 of the spacer and cannot occur when the LoxPsym sites comprise a different nucleotide on position 2, 3, 6 and/or 7 of the spacer, except for the spacer combinations GGGTACCC-AACTAGTT, GGGTACCC-ATATATAT, GAATATTC-AGTTAACT, GTGTACAC-GCATATGC.

In another embodiment, the orthogonal LoxPsym site is selected from any of the sets of LoxPsym sites herein disclosed, more particularly selected for a set of LoxPsym sites comprising at least two LoxPsym sites having the following formula: 5′-ATAACTTCGTATA—spacer—TATACGAAGTTAT-3′, wherein the spacer is selected from the list consisting of SEQ ID No. 1-63, and wherein the at least two LoxPsym sites comprise a different nucleotide on position 2, 3, 6 and/or 7 of the spacer, and wherein the set does not comprise the LoxPsym sites depicted in SEQ ID No. 88 and 89, SEQ ID No. 88 and 81, SEQ ID No. 90 and 83, SEQ ID No. 65 and 92, or SEQ ID No. 78 and 103.

In a particular embodiment, the orthogonal LoxPsym site is selected from SEQ ID No. 65, 69-71, 74, 77, 82, 84, 100, 105, 107-109, 112, 121 and/or 124 or selected from the list consisting of SEQ ID No. 65, 69-71, 74, 77, 82, 84, 100, 105, 107-109, 112, 121 and 124.

In some embodiments of the methods herein described, the activity of the recombinase is stopped by manipulating the in vitro reaction by application of heat or a denaturing or chelating agent. In particular embodiments of the invention, the methods herein provided include an additional step of introducing a recombined polynucleotide into a cell, more particularly a plant cell, an insect cell, a mammalian cell or a microorganism to obtain a genetically modified cell, and determining the function of the recombined polynucleotide by analysis of the genetically modified cell. In particular embodiments, the modified cell is a microorganism, even more particular a bacterium or a yeast, most particular a Saccharomyces, Yarrowia or Pichia yeast. In another particular embodiment said modified cell is a cell of a species selected form a group consisting of Saccharomyces cerevisiae, Escherichia coli, Zea mays and Yarrowia lipolytica. In particular embodiments, the methods herein provided include an additional step of determining the sequence of the recombined polynucleotide, and/or one or more functions of proteins or functional RNAs encoded by the recombined polynucleotide.

The application also provides a recombinant polynucleotide made by any method described herein, and cells, more particularly microorganisms, such as yeast or bacteria, comprising any polynucleotide made by a method described herein.

In certain approaches the disclosure comprises the use of the Cre-LoxPsym systems herein described in combination with one or more other recombination systems selected from the list consisting of Flp Recombinase which functions in the Flp/FRT system, the Dre recombinase which functions in the Dre-rox system, the Vika recombinase which functions in the Vika/vox system, Bxb 1 recombinase which functions with attP and attB sites, long terminal repeat (LTR) site specific recombinase (Tre), and other serine recombinases, such as phiC31 integrase which mediates recombination between two 34 base pair sequences termed attachment sites (att), Hin recombinase, which recognizes 26 bp imperfect inverted repeat sequences or int2-13 each of which each recognizes distinct target sites of 39-66 bp.

The application also provides a cell line comprising a plurality of landing pads integrated into the genomic DNA of a parental cell line. The parental cell line may be a wild type cell line, or a cell line with existing genomic modification. In the latter, the cell line would be “parental” to the cell line generated from further modification of its genomic DNA. A “landing pad” is an exogenous DNA sequence integrated into a location of the host genome that includes a LoxPsym site. In some instances, the exogenous DNA sequence includes a LoxPsym site, a constitutive promoter operably linked to a nucleotide sequence encoding a detectable marker, followed by a nucleotide sequence encoding a first selectable marker. In certain types of landing pads, the LoxPsym site is between the promoter and the nucleotide sequence encoding the detectable protein. When there are more than one landing pads used in a given cell, it is preferred that a LoxPsym site of one landing pad is orthogonal to a LoxPsym site in any other landing pad. The landing pad is used for further genetic engineering and integration of a nucleic acid molecule of interest via site-specific recombination. The landing pad can be integrated into the parental genome using any method known in the art, such as by using a zinc finger nuclease, TALEN, or the CRISPR-Cas system. In some embodiments, the number of landing pads integrated into a cell line is 2, 3, 4, 5, 6, 7, 8, 9, 10, 11, or 12. In some embodiments, the LoxPsym site in the landing pad is selected from SEQ ID No 65-127, most particularly selected from SEQ ID No. 65, 69-71, 74, 77, 82, 84, 100, 105, 107-109, 112, 121 and/or 124. In some embodiments, the detectable marker in the landing pad is a fluorescent protein, such as, eGFP, eYFP, eCFP, mKate2, mCherry, mPlum, mGrape2, mRaspberry, mGrapel, mStrawberry, mTangerine, mBanana, and mHoneydew, luciferase, or LacZ. In some embodiments, a selectable marker hydrolyzes a drug, such as, puromycin, hygromycin, G418, neomycin, or bleomycin.

Further provided herein, is a method of integrating a genetic circuit, or multiple genetic circuits, into a cell comprising a plurality of landing pads. In some embodiments, one genetic circuit is integrated into the cell line. In some embodiments, 2, 3, 4, 5, 6, 7, 8, 9, 10, 11, 12 or more circuits may be integrated into the cell line, provided that the number of the landing pads in the cell line is sufficient to accommodate the number of genetic circuits that are to be integrated into the cell line. In some instances, it may be preferred that the number of landing pads is at least the number of genetic sequences or circuits to be integrated. In other instances, a single landing pad can include multiple circuits under the control of different promoters. A “genetic circuit”, as used herein, is a rationally designed artificial gene regulatory networks with robust function, comprising primary genetic elements or building blocks. Non-limiting examples of primary genetic elements are promoters, ribosome binding sites, transcriptional activators and repressors, gene coding sequences, 5′UTRs, 3′UTRs, polyA signals, and terminators. Independent modules of a genetic circuit can be built using the primary genetic elements. Methods of building these genetic circuits are known to those of skill in the art. In some embodiments, a plurality of landing pads may be integrated into different locations of the genome, allowing modification at multiple loci of the genome via site-specific recombination.

Additional to the above detailed description of the invention, the following experimental details further enable the skilled person to put all details of the invention into practice.

### EXAMPLES

### Example 1. New LoxPsym Recombination Sites Strongly Influence Recombination Efficiency in Saccharomyces cerevisiae

Similar to the development of the original LoxPsym site by Hoess et al. (1986 J Cell Biochem), we edited the spacer of the LoxP site to obtain new non-directional recombination sites. In doing so, we focused on altering the first three nucleotides of the spacer (FIG. 2), as it was shown before that changing the nucleotides at position 4 and 5 of the spacer (T and A respectively) prevents recombination (Hoess et al 1986). The last three nucleotides were adapted accordingly to ensure the spacer remained palindromic. As such, we were able to design maximally 63 new potential LoxPsym sites. To test if the Cre enzyme was still able to recombine the new LoxPsym variants, we conducted a fluorescent-based assay in yeast via genomic integration of a fluorescence cassette which was flanked by identical LoxPsym variants at both sites (FIGS. 3A and 3C). Recombination of functional LoxPsym variants enables the deletion of the fluorescence cassette. The fraction of non-fluorescent cells in the population after induction of Cre-mediated recombination, was used to determine the recombination efficiency of the new LoxPsym sites. To verify if this set-up is valid for estimation of the recombination efficiency, the dual-fluorescent-reporter system shown in FIG. 3A was used to assess the frequency of inversion/deletion over time. Measurements after 0, 6, 10, 24 h of induction demonstrate that the frequency of inversions is negligible compared to deletions over time, hence indicating that the reporter system for determination of recombination efficiencies is valid (FIG. 3D). Recombination efficiencies largely altered between different LoxPsym variants (Table 1). 63 new LoxPsym variants were obtained with altering recombination efficiencies ranging from 13% to 89%. Interestingly, 36 new LoxPsym sites displayed recombination efficiencies which were higher than that of the original LoxPsym site currently used by the research community (Hoess et al. 1986, Richardson et al. 2017), reaching a 1.9-fold increase in observed recombination events (Table 1).

Finally, it was investigated if the purine/pyrimidine-content of the spacer or the distribution of purines/pyrimidines within the spacer region could explain the large diversity in recombination efficiencies amongst the LoxPsym variants, but no clear relationship could be observed (FIG. 3E).

### Example 2. A Large Set of Orthogonal Recombination Sites could be Identified

After confirming that the newly developed LoxPsym sites enable recombination in yeast, we next aimed to identify orthogonal LoxPsym sites. As the spacer is the target for cutting and strand exchange during Cre-mediated recombination, we reasoned that non-homologous spacers would prevent recombination. Therefore, a similar assay as described previously was performed, but in this case the LoxPsym sites which flanked the fluorescence cassette were different up- and downstream of the fluorescence marker (FIG. 3B-3C). To enable the identification of a large number of orthogonal sites, we assessed the interactions between 48 LoxPsym variants. Specifically, this set contained all variants which had nucleotide T, A or G at the first position of the spacer. This way, an additional 1008 S. cerevisiae strains were constructed in order to assess the interaction between different LoxPsym variants and the recombination efficiencies are depicted in FIG. 3G. The high recombination efficiencies shown at the diagonals in the matrix indicate that recombination occurs between identical sites, as well as between non-identical sites that differ only in the first and last (because of the palindromic nature of the LoxPsym site) nucleotide of the spacer. Interestingly, it can be seen that these nucleotides still play a role in the occurrence of cross-reactivity between variants that differ at the other positions of the spacer. For instance, where recombination between the LoxPsym variants with spacer sequences AGTTAACG-GAATATTC is occurring, recombination between LoxPsym spacers AGTTAACG-TAATATTA is prevented, thus indicating that the first nucleotide of the spacer cannot simply be neglected in the screen. Most importantly, combining all data, we were able to identify a large set of 16 orthogonal LoxPsym variants which are still functional but do not interact with each other. Finally, to verify the interactions between non-identical LoxPsym variants, some of the strains in which a deletion of the fluorescence cassette occurred, were analyzed via sanger sequencing (FIG. 3H). The sequence of the remaining LoxPsym site shows that a mismatch at the first and last nucleotide of both spacers consistently yields a hybrid LoxPsym variant, that contains traces from both parent sites and is therefore no longer completely symmetrical. Mismatches between LoxPsym variants in the middle of the spacer did not result in hybrid scars, rather the sequence of only one of the two original LoxPsym sites can be found.

### Example 3. Applying the Expression Optimisation Tool on the Astaxanthin Production Pathway in Yeast can Enhance Strain Performance

As one application of the orthogonal LoxPsym sites developed in this study, the new variants were used for shuffling genetic promoter and terminator elements. First, it was assessed if a combination of several repeats of 2 orthogonal LoxPsym sites (either in the promoter or terminator construct) allows to diversify gene expression upon induction of the Cre recombinase. This was done by targeting a fluorescent reporter (yECitrine) and analysing several single clones after recombination (FIG. 4A-violin plots). It was observed that expression can be altered by shuffling both promoters and terminators. Sequencing confirmed that this was done without cross-reaction of the 2 orthogonal LoxPsym variants used (specifically spacer SEQ ID No. 6 and 20 and SEQ ID No. 70 and 84) (FIG. 4B-sequencing results).

Next, a combination of 12 orthogonal LoxPsym variants (SEQ ID No. 69, 70, 74, 77, 84, 86, 88, 100, 108, 109, 114 and 124) was used to alter the expression of 6 genes of the astaxanthin production pathway in S. cerevisiae (genes showed above arrows in FIG. 5A). Induction of recombination causes color variation of the strains, due to altered production of the intermediate carotenoids (such as lycopene, beta-carotene and zeaxanthin) (FIG. 5B-C). Several single clones were further analyzed on carotenoid content (FIG. 5D, 5F) and gene expression levels of the 6 genes of interest (FIG. 5E). This application therefore demonstrates that multiple orthogonal sites can applied simultaneously for the in vivo optimisation of the expression of several genes simultaneously. This allows to optimize the phenotype of interest, with a >2-fold increase in astaxanthin production in this particular example.

### Example 4. A Set of Orthogonal LoxPsym Variants can be Applied for Multiplex Genome Engineering

The capability of simultaneously using all 16 orthogonal LoxPsym variants that were identified in the previous assays was further investigated. More in detail, the application of all 16 sites present in the same genome, with maintained orthogonality, was analyzed, since this is an absolute requirement for applying these recombination sites, for example, to facilitate complex metabolic engineering efforts where typically several genomic loci are altered for gene insertion or deletion, either simultaneously or consecutively. Our test relied on 18 constructs, including 16 test constructs that each assessed the functionality of one LoxPsym variant in the presence of all other variants, and 2 controls (FIG. 6A). To select for recombination in all cases, all constructs used the deletion of URA3 (resulting in tolerance towards 5-fluoroorotic acid; FOA) by surrounding the marker with two LoxPsym-TCA sites and plating on SC+FOA. Additionally, to determine efficiency of each specific LoxPsym site in the presence of all other sites, each construct encoded an ADE2 marker. The deletion of this marker results in a red pigmented yeast colony and allows the detection of a second recombination event. The two control constructs (differing in the location of the ADE2 marker) exhibited only one copy of each LoxPsym site (in exception of LoxPsym-TCA, which allows for selection of recombination positive clones) and deletion of ADE2 was not expected. In contrast, each test construct included one extra copy of one specific LoxPsym variant upstream of the ADE2 marker and deletion of ADE2 was expected and used as a read-out of the recombination efficiency of that site (FIG. 6B-6C). LoxPsym-CAC showed a very low recombination rate (0.9116±0.6606%), indicating recombination of this LoxPsym variant was reduced by the presence of the 15 other sites. The other LoxPsym variants showed higher activities, although the recombination efficiencies were consistently lower and did not correlate well with previously calculated ones, which may be the results of differences in experimental setup. In particular, our results suggest that the genomic context of the sites plays a major role because the two sites showing the highest efficiency (LoxPsym-TTA and -TCA), were located at both edges of the LoxPsym array. Moreover, a negative correlation (R²=0.37, p-value=0.013) can be observed between the efficiency and the distance to the edge of the LoxPsym array, indicating that the efficiency drops because more LoxPsym variants hinder the recombination site of interest to be bound or find its correct interaction partner. We reason the cause underlying this observation is a combination of the correlation between the recombination efficiency and the distance between interacting recombination sites (Hoess et al 1985 Gene 40:325-329, Zheng et al 2000 Molec. Cell. Biol. 20:648-55), the reduced ratio of Cre enzymes and its target site and the formation of nonproductive synapses between incompatible recombination sites, which could shield the recombination sites from recombining with the functional interaction partner (Lee and Saito 1998 Gene 216:55-65, Fan 2012 Nucleic Acids Res. 40:6208-6222).

In addition, the design of the constructs allowed calculating the frequency of cross-reactivity by PCR, since recombination between different LoxPsym sites would result in reporters with a different length (FIG. 6). The measured PCR fragment length deviated from the expected size in only 9 out of the 208 randomly picked red colonies (13 per test construct), indicating some level of cross-reactivity. To further assure that the recombination events corresponded with the expected patterns, we sequenced three PCR fragments per test construct and observed the expected result in all but one case.

### Example 5. LoxPsym Variants are Also Functional and Orthogonal in Prokaryotes and Higher Eukaryotes

Besides the identification and multiplexing of the set of 16 orthogonal LoxPsym variants in yeast, we further analyzed the potential to of these novel recombination sites as functional and orthogonal tools in other species, specifically Escherichia coli and Zea mays. For assessment of the functionality and cross-reactivity of the LoxPsym variants in E. coli, we set up a plasmid-based assay testing pairwise combinations between 16 different donor and acceptor plasmids, each carrying one LoxPsym variant (FIG. 8A-8B). After inducing recombination, cross-reactivity between LoxPsym variants was detected via PCR amplification of the junction that spanned the recombined recombination site. All tested

LoxPsym variants showed recombination activity in bacterial cells, although no correlation was observed with the activity of the respective sites in yeast and plants (FIG. 8C). In contrast to the data obtained for S. cerevisiae, we did observe cross-reactivity in a few cases. Sequencing of these recombined scars revealed up to three mutations in LoxPsym sites resulting from recombination between cross-reactive partners. Actually, it has previously been observed that results indicating orthogonal recombination are not always transferrable between pro- and eukaryotes, which may be caused by a slightly altered protein structure or activity of the recombinase in different host organisms, or differences in native cellular processes, such as the involved DNA mismatch repair pathway to restore mismatches that appear after recombination. Additionally, the difference in experimental set-up can also be an explanation for the observed differences: recombination (deletion) was irreversible for the experiments in yeast, whereas recombination of the two plasmids was reversible in bacteria. Importantly, the cross-reactivity was much lower than the recombination activity observed between identical LoxPsym sites.

For the characterization of cross-reactivity in higher eukaryotes, we used Zea mays, one of the most important cereal crops with a widespread use in food and feed, as well as in industrial applications. We performed a plasmid-based assay using maize mesophyll protoplasts. Briefly, we constructed a library of plasmids which contain two LoxPsym sites separated from each other by a short linker that incorporated two restriction sites to digest this plasmid later in the workflow (FIG. 7A-7B). Each LoxPsym variant was accompanied by a unique barcode allowing the identification of which LoxPsym pair was involved in the recombination process by sequencing barcodes surrounding the recombined LoxPsym site. A combinatorial cloning scheme was used, and all 256 combinations of the 16 LoxPsym variants were present in the final plasmid pool and confirmed by NGS sequencing. The plasmid pool and a plasmid constitutively expressing the Cre recombinase or an empty backbone were co-transfected into maize mesophyll protoplasts and the region spanning the LoxPsym site(s) was amplified by PCR 48 h after transfection. Recombination was only detected in the presence of the Cre recombinase and this reaction was send for NGS sequencing to assess the efficiency of recombination of each LoxPsym pair by identifying barcode frequencies in the pool (normalizing to abundance in the starting pool) (FIG. 7C). The results confirmed activity of recombination in the higher eukaryote Z. mays, and showed the absence of cross-reactivity between selected LoxPsym variants. Moreover, a broad range of recombination efficiencies linked to different LoxPsym sites was detected, albeit with a weak correlation to the efficiencies that we observed in yeast (R²=0.02, p-value=0.6056).

In conclusion, we assessed cross-reactivity between a selection of the LoxPsym variants by a pairwise interaction assay and identified a set of 16 orthogonal LoxPsym variants that can be used simultaneously with no or only minimal cross-reaction. We demonstrated that the sites described herein can also be used in other species beyond yeast, including E. coli and Z. mays. Together, these findings dramatically expand the potential of using Cre-LoxPsym as a gene editing technology, especially for cases where recurrent and/or multiplexed recombination is desirable, for example during strain construction in metabolic engineering efforts.

### Example 6. LoxPsym Variants are Also Functional and Orthogonal in Y. lipolytica

To prove that the alternative LoxPsym sites (Table 2) are orthogonal in Y. lipolytica we transformed the wild type strain W29 with a construct consisting of the 16 LoxPSym sites separated by a spacer sequence (100 bp). The construct has a total length of 2284 bp and was targeted at the URA3 locus (FIG. 9A). After the correct integration of this construct the generated strain was subsequently transformed with a Y. lipolytica replicative plasmid containing an expression cassette for the Cre recombinase (codon optimized for S. cerevisiae) and the NAT selectable marker conferring resistance to neomycin (FIG. 9A). The resistant colonies were then screened with PCR for Cre-mediated recombination. The primers used are called 246-F (TGGTTTAGTGTATGTTGCGC) and 247-R (CTAAGTCTGTGCTCCTTCC) and they are flanking the constructs with the 16 LoxPSym sites. If the sites are orthogonal then Cre-mediated recombination will not occur and the primers will amplify a single product of 2284 bp. In case that some of the LoxPsym sites cross-react then after the PCR screening smaller bands of variable sizes will appear depending on the specific lox sites that have recombined.

In total a number of 192 transformants was screened after the transient expression of Cre recombinase. In all the cases a single band of ~2.3 kb was amplified (FIG. 9B), indicating that the alternative lox sites are orthogonal in Y. lipolytica.

One of the most common applications of the Cre/lox system is marker recycling. In this, a selectable marker is flanked by the LoxP site and after the transient expression of the Cre recombinase the selectable marker is looped out and can be used for a next round of transformation. This Cre-mediated recombination leaves behind a LoxP site that in the next round can cross-react with the newly inserted LoxP sites resulting in genetic rearrangements (Steensels et al 2018, Nat Commun 9:1937). To further test if the new LoxPsym sites are orthogonal and can be used for multiple rounds of marker curation without the risk of SCRAMBLEing the genome (Steensels et al 2018, Nat Commun 9:1937), we checked the efficiency of looping out the selectable marker hph, conferring resistance to hygromycin, using 4 different LoxPsym sites (IoxP, LoxPSym 0, LoxPSym 2, LoxPSym 4; FIG. 10A; Table 2). We repeated these experiments 3 times and every time 192 colonies were checked for resistance to hygromycin (by growing them on plates supplemented with 100 g/ml hygromycin B). The average frequency of marker excision was 81.9% for LoxPSym 0, 80.7% for LoxPSym 2, 80.6% for LoxPSym 4, and 793% for IoxP (FIG. 10B). The results show that alternative LoxPSym are orthogonal with high frequency of Cre-mediated recombination, and thus can be combined without the risk of cross-reactivity when multiple rounds of genetic engineering and subsequent marker recycling are needed.

TABLE 2
Orthogonal LoxPsym sites in Yarrowia lipolytica.

| Label of LoxPSym orthogonal | |
|---|---|
| site in FIGS. 9 and 10 | Sequence |
| LoxPSym 0 | SEQ ID No. 70 |
| LoxPSym 2 | SEQ ID No. 84 |
| LoxPSym 4 | SEQ ID No. 108 |
| LoxPSym 6 | SEQ ID No. 69 |
| LoxPSym 7 | SEQ ID No. 109 |
| LoxPSym 3 | SEQ ID No. 100 |
| LoxPSym 9 | SEQ ID No. 71 |
| LoxPSym 21 | SEQ ID No. 65 |
| LoxPSym 17 | SEQ ID No. 121 |
| LoxPSym 6b | SEQ ID No. 82 |
| LoxPSym 30 | SEQ ID No. 112 |
| LoxPSym 24 | SEQ ID No. 74 |
| LoxPSym 19 | SEQ ID No. 105 |
| LoxPSym 18 | SEQ ID No. 124 |
| LoxPSym 25 | SEQ ID No. 107 |
| LoxPSym 15 | SEQ ID No. 77 |

### Materials and Methods

### Molecular Methods.

DNA amplification was done by PCR using SapphireAmp Fast PCR mix (Takara Bio), Phusion (NEB) or GXL (Takara Bio) DNA polymerase. DNA oligonucleotides were obtained from Integrated DNA Technologies (IDT). Synthesis of longer DNA constructs was ordered from Qinglan Biotech, BGI. The pV1382 backbone (Addgene Plasmid #111436) was used to express sgRNA, which was ligated into the BsmBI-digested backbone after annealing of the oligonucleotides, as previously described (Vyas et al 2018 mSphere 3: e00154-1). Plasmids reported in this study were constructed using Gibson Assembly (NEBuilder HiFi DNA Assembly Master Mix) for plasmids used in E. coli and S. cerevisiae, and using Golden Gate cloning (GreenGate cloning standard reported by Lampropoulos et al 2013 PLOS One 8: e83043) for plasmids used in Z. mays. Purification of plasmids needed for experiments in yeast and bacteria were purified using the QIAprep Spin Miniprep Kit (Qiagen). Purification of plasmids needed for experiments in plant cells were done using the ZymoPURE II Plasmid Midiprep Kit (Zymo Research). Sanger sequencing was performed by Eurofins Genomics.

### Strains and Growth Conditions.

E. coli strains were constructed from the lab strain DH5α (NEB) and cells were grown in Luria Bertani (LB) medium (10 g/L peptone, 10 g/L NaCl, 5 g/L yeast extract) at 37° C., shaking at 200 rpm. Antibiotics (chloramphenicol, carbinicilin and kanamycin) were added at 50 g/mL. Inducer L-Rhamnose was added at 2%. S. cerevisiae strains were constructed from the lab strain BY4741, which is an S288C-derivative laboratory strain with genotype MATa his3Δ1 leu2Δ0 met15Δ0 ura3Δ0. Cells were grown in Synthetic Complete (SC) medium (0.79 g/L SCM, 6.7 g/L YNB) or SC-Histidine medium. Carbon sources (glucose, raffinose and galactose) were added at 2%. Z. Mays protoplasts (cv. B104) were isolated as previously described and suspended in W5 solution, see below (Gaillochet et al 2023 Genome Biol. 24:6).

S. cerevisiae Transformation Protocol.

1 mL overnight (ON) grown culture in 2×YPD (20 g/L yeast extract, 40 g/L peptone, 4 g/L glucose) was inoculated into 50 mL 2×YPD for 3 h. Cells were centrifuged (3 min, 3000 rpm) and consecutively washed with 10 mL and 1 mL 0.1 M lithium acetate (LiOAc). Cells were resuspended in 100 μL 0.1 M LiOAc. PCR amplified donor DNA (50 μL) and/or plasmid DNA (200 ng) were added. CRISPR/Cas9 was used for genomic DNA insertion using pV1382 with inserted gRNA of interest. A mixture containing 620 μL 50% PEG 3350, 4 L salmon sperm DNA and 90 μL 1M LiOAc was added and mixed by vortexing. Cells were incubated for 30 min at 30° C., 400 rpm. 100 μL DMSO was added prior to a 15 minutes heat shock at 42° C. Cells were harvested by centrifugation (3 min, 3000 rpm) and washed with 5 mM CaCl2). Cells were incubated for a 3 h recovery period at 30° C., 400 rpm and finally plated on selective medium. Colony PCR (SapphireAmp Fast PCR Master Mix, TakaRa) using a template prepared by boiling the clone in 50 μL NaOH (0.02 M) (99° C., 10 min) to amplify the junction of desired insertion was used to identify positive transformants.

Fluorescence Assay and Recombination Induction in S. cerevisiae.

Strains were derived from BY4741 with constitutive expression of fluorescent reporter mCherry (Smukalla, S. et al 2008 Cell 135:726-737). To test the LoxPsym variants, strains carried an overexpressed yECitrine reporter gene, which was regulated by the TDH3 promoter and CYC1 terminator, flanked by two LoxPsym variants (inserted via LoxPsym-tailed primers) and genomically integrated at the CAN1 locus. Single colonies were inoculated in 100 μL SC-His 2% glucose for ON growth. Cells were washed and diluted in SC-His 2% raffinose to a final OD 0.05 and grown ON. Cells were washed and diluted in SC-His 2% raffinose 2% galactose for induction of Cre expression from the control backbone (without Cre) and the plasmid with the pGAL1-Cre expression cassette. Cells were induced for 6 h, unless indicated otherwise. Cells were washed and diluted to SC 2% glucose for ON recovery (dilution 1/20), after which cells were plated on YPD and/or used for flow cytometry analysis.

### Fluorescence Analysis.

Flow cytometry was performed using the Attune NxT Flow Cytometer and Auto Sampler. Cultured yeast cells were diluted in focusing fluid and measured with a flow rate of 200 μL/min. Cytometry data was gated based on the FSC-H to FSC-A map to select for single cells. For determination of the recombination efficiency, an additional gating was performed using the control fluorescent reporter mCherry (mCherry+ cells were selected for further analysis). yECitrine and mCherry were measured using channels BL1-A (excitation at 488 nm and emission at 574 nm with 20 nm bandwidth) and YL2-A (excitation at 561 nm and emission at 610 nm with 20 nm bandwidth), respectively. Analysis and gating steps were done using the FlowJo software with (non-) fluorescent control strains as a reference. Recombination efficiencies lower than the reference were set to 0 to remove noise from the data. To determine yECitrine fluorescence of single clones, single colonies were inoculated in SC 2% glucose and fluorescence was measured using plate reader (TECAN Infinite 200 Pro), using excitation at 498 nm with bandwidth 9 nm and emission at 535 nm with bandwidth 20 nm. Data was obtained after normalization by the absorbance at 600 nm. Division into fluorescent/non-fluorescent groups was done by comparison with values obtained for control strains.

Multiplexed-LoxPsym Assay and Recombination Induction in S. cerevisiae.

Strains were derived from BY4741 with a deletion of pADE2-ADE2-tADE2, constructed using sgRNA3, with test and control (FIG. 6A) inserted at the CAN1 locus and either P1 (Cre) or P2 (control). Induction of recombination similar to methods described above. After ON recovery in SC 2% glucose, cells were plated on SC and SC+FOA and incubated for 48 h at 30° C., after which colonies were counted for each plate. Red colonies were selected for PCR amplification of the recombined constructs. Length of the amplicons was determined using capillary electrophoresis (QIAxcel Advanced instrument, QIAxcel DNA Screening Cartridge, QX Size Marker 250 bp-4 kb v2.0) to visualize small differences in band length.

E. coli Transformation Protocol.

For heat shock transformation, chemically competent E. coli cells were thawed on ice for 30 minutes. Plasmid DNA (50-100 ng) or 2 μl of the Gibson/Golden Gate reaction was mixed with 25 μl of competent cells in an ice-cold 1.5 mL Eppendorf tube. After 30 minutes incubation on ice, the reaction was heat shocked for 30 seconds at 42° C. and chilled on ice for 5 minutes. A volume of 300 μL SOC medium was added, and the tube was incubated at 37° C. for 60 minutes in a shaking incubator. Finally, 100 μL of cells was plated on pre-warmed (37° C.) LB medium containing the appropriate antibiotics and incubated at 37° C. for ON growth. For electroporation, we used commercial NEB 10B cells (NEB) with a transformation efficiency of 2×1010 cfu/μg. 2 μL of the assembly reaction was mixed with 50 μL of competent cells and placed inside a chilled electroporation cuvette (0.2 cm gap, BioRad). The electroporation was carried out in a GenePulser (BioRad) according to the manufacturer's conditions and 900 μL of SOC medium was added immediately to the cells afterwards. Cells were incubated at 37° C. for 60 minutes in a shaking incubator. Finally, 100 μL of cells were plated per pre-warmed (37° C.) LB plate containing the appropriate antibiotics.

Recombination Assay in E. coli.

Bacterial strains were derived from DH5a after co-transformation of acceptor and donor plasmids using double selective medium LB+kanamycin (Kan)+chloramphenicol (Cm). Single colonies were inoculated in 100 μL LB+Kan+Cm for ON growth. Cells were washed and diluted (1/20) in LB 2% rhamnose+Kan+Cm for induction of Cre expression from acceptor plasmids (under control of the rhaB promoter). After 4 h induction, cells were washed and grown ON in LB+Kan+Cm. Recovered cells were harvested by centrifugation (3500 rpm, 5 min) and suspended in dH₂O. Cells were boiled for 10 minutes at 99° C. and the remaining mixture was used as a template for PCR to amplify the junction of recombined donor and acceptor plasmids. We reasoned that amplifying one of the two recombined junction possibilities (donor plasmid could insert in two directions in the acceptor plasmid) was sufficient, as recombination between symmetrical sites should not favor one of both options and the combination of two independent plasmids avoided the accumulation of one recombination outcome. Amplicons were subjected to densitometric analysis using the Image J software to extract peak areas (from a plot of the lanes). Peak areas of the junction were normalized by division with areas extracted from the most abundant control amplicon (derived from PCR performed on separate donor and acceptor plasmids).

Combinatorial LoxPsym Library Construction for Assay in Z. mays.

For construction of the LoxPsym combinatorial library, we applied Golden Gate cloning using the GreenGate cloning standard (Lampropoulos et al 2013 PLOS One 8: e83043) to assemble 5 entry clones. Entries A and E were constructed by ligating annealed oligonucleotides into BsaI-digested entry vectors pGGA000 (Addgene #48856) and pGGE000 (Addgene #48860), resp. The entries for the 16 barcode-LoxPsym combinations in position B and D were made in the same manner using oligonucleotides. The linker at position C was PCR amplified from the pUC19 plasmid (Addgene #50005). After gel purification using the Zymoclean Gel DNA Recovery Kit, the purified product was combined with pGGC000 (Addgene #48858) in a Gibson assembly reaction using NEBuilder master mix (NEB). For the final Golden Gate reaction of the LoxPsym combinatorial library, all entries were pooled and entries B and D contained a mix of all LoxPsym variant plasmids at equal concentrations (16 plasmids/position; quantified with Qubit™ dsDNA HS assay) to make a combinatorial library of plasmids containing the 256 different LoxPsym combinations, which was next transformed to DH10B cells. After overnight incubation, the colonies of nine different plates (>50000 colonies) were scraped and suspended in LB medium. The plasmid DNA was extracted with the ZymoPURE II Plasmid Midiprep Kit (Zymo Research). Plasmids were diluted to 1 μg/μL. The plasmid expressing Cre recombinase was also constructed using Golden Gate, starting from available parts (and was purified and diluted similarly.

Z. mays Protoplast Isolation and Transfection.

The isolation and transfection of maize protoplasts was performed as previously described (Gaillochet et al., 2023 Genome Biol. 24:6). Transfections were done in 1 mL strip tubes (TN0946-08B, National Scientific Supply Co), using 100 μl of protoplasts (10⁵ cells), 110 μL of PEG solution (0.2 M mannitol, 100 mM CaCl2)) and 40% PEG 4000 together with 20 μg of plasmid DNA (10 μg of the combinatorial IoxP plasmid library and 10 μg of the control or Cre expression plasmid). Each transfection was done in triplicate. The protoplasts were suspended in W5 solution and incubated in 24-well plates in the dark at 25° C. on a shaking platform (20 rpm). Samples were harvested after two days and stored at −20° C. until further processing.

Z. mays DNA Extraction.

A modified Edwards extraction protocol was used for the isolation of protoplast DNA (Edwards et al 1991 Nucleic Acids Res. 19:1349). The extraction buffer was composed of 100 mM Tris HCl (pH 8), 500 mM NaCl, 50 mM EDTA and 0.7% SDS. Protoplasts were transferred to 1.5 mL Eppendorf tube and were spun down at 12000 rcf for 5 minutes, after which the supernatant was removed. A volume of 200 μL extraction buffer was added to the Eppendorf tubes and the tubes were manually shaken to dissolve the pellet. After 15 minutes of incubation at 60° C., the tubes were cooled down to room temperature. A volume of 200 μL 100% isopropanol was added and the tubes were spun down at 12000 rcf for 10 minutes. The supernatant was removed, and the pellet was washed with 200 μL 80% ethanol. After air drying for 15 minutes, the pellet was dissolved in 20 μl of 10 mM Tris-HCl pH 8 (preheated at 60° C.). After incubation of the tubes in a 60° C. thermoblock for 10 minutes, the tubes were stored at −20° C. until further processing.

### Next Generation Sequencing.

For sequencing of the input plasmid library for Z. mays transfection, we set up a 40 μL PCR reaction with the Phire Plant Direct PCR Kit (Thermo Scientific) using 4 μL of the diluted midiprep (100 ng/μL) as the template and primers OF/R82. The following PCR conditions were used: 98° C./2 min+10× (98° C./5 sec+62° C./5 sec+72° C./10 sec)+72° C./2 min+23° C./∞. The fragment of the correct size (approx. 270 bp) was purified using the Zymoclean Gel DNA Recovery Kit according to the manufacturer's instructions. A similar set-up was used for the sequencing of the protoplast assay fragments, using 4 μL of the protoplast DNA as the template and primers with different demultiplexing tags for each sample in a total reaction volume of 40 μL. The PCR conditions used were as follows: 98° C./2 min+25×(98° C./5 sec+62° C./5 sec+72° C./10 sec)+72° C./2 min+23° C./∞. We could not detect any evidence of recombination in our agarose electrophoresis results and reasoned that this could be due to the massive amount of plasmid DNA that was transfected (~32 million plasmid copies per protoplast). Therefore, we used restriction-digestion of the extracted DNA to specifically cut the C-linker-D module of the non-recombined plasmids to bias against amplification of these DNA species. Digestion of the protoplast DNA was done with NcoI-HF (NEB) and PvuI-HF (NEB) in CutSmart buffer for 12 hours at 37° C. Amplicons were constructed using primers OF/R83-88 purified with the GeneJET PCR Purification Kit (Thermo Fisher) according to the manufacturer's instructions. Samples were sent to Eurofins (Germany) for adapter ligation and NGS sequencing (5 million paired reads, 2×150 bp). For each plasmid, the number of reads detected for the protoplast DNA was normalized by the number of reads present in the input library.

Yarrowia lipolytica Strain and Culture Conditions.

The reference strain Y. lipolytica W29 was used in all the experiments mentioned. The yeast was grown in Yeast Extract-Peptone-Dextrose (YPD) at 30° C./220 r.p.m (when liquid cultures were used) for 2-3 days. The selective reagents were added at the following concentrations: nourseothricin (CloNAT), 220 g/ml; hygromycin B 100 μg/ml.

Yeast (Y. lipolytica) Transformation.

Transformation of the Y. lipolytica was performed following an already described protocol (Abdel-Mawgoud and Stephanopoulos 2020, Metab Eng 62:106-115), with minor modifications.

### Colony Picking and PCR Screening.

Transformed colonies were picked from the transformation plate and pinned to the selection plates using the PIXL Precision Microbial Colony Picker (Singer Instruments). Colony PCR screening was performed with the SapphireAmp fast PCR polymerase (Takara Bio), using the primer pair 246-F/247-R.

1. A LoxPsym site comprising the following formula: 5′-ATAACTTCGTATA—spacer—TATACGAAGTTAT, wherein the spacer is selected from the group consisting of SEQ ID Nos. 1-63.

2. A The LoxPsym site of claim 1, wherein the recombinase Cre cleaves the LoxPsym site.

3. A The LoxPsym site of claim 1, the LoxPsym site further comprising a nucleic acid sequence selected from the group consisting of SEQ ID Nos. 65-127.

4. A The LoxPsym site of claim 1, wherein the LoxPsym site has a recombination efficiency that is lower than that of a standard LoxPsym site as set forth in SEQ ID No. 128 under the same conditions and wherein the LoxPsym site comprises a spacer selected from the list consisting of SEQ ID No. 1-27.

5. A The LoxPsym site of claim 1, wherein the LoxPsym site has a recombination efficiency that is higher than that of the standard LoxPsym site as depicted in SEQ ID No. 128 and wherein the LoxPsym site comprises a spacer selected from the group consisting of SEQ ID Nos. 28-63.

6. The LoxPsym site of claim 1, wherein, in the presence of recombinase Cre, a specific DNA recombination cannot occur between the LoxPsym site and a second LoxPsym site according to claim 1 and wherein the LoxPsym site and the second LoxPsym site comprise a different nucleotide on positions 2, 3, 6 and/or 7 of the spacer.

7. The LoxPsym site of claim 6, wherein the LoxPsym site and the second LoxPsym site do not comprise the spacers GGGTACCC and AACTAGTT, GGGTACCC and ATATATAT, GAATATTC and AGTTAACT, GAGTACTC and AAATATTT, or GTGTACAC and GCATATGC.

8. The LoxPsym site claim 6, wherein the LoxPsym site is selected from the group consisting of SEQ ID Nos. 65, 69-71, 74, 77, 82, 84, 100, 105, 107-109, 112, 121 and 124; and/or selected from the list consisting of SEQ ID No. 70, 77, 82, 101-102, 105, 107, 109, 112, 114, 117-118, 123-124, 126 and 127; and/or selected from the list consisting of SEQ ID No. 65, 71-74, 84, 86, 88, 90, 103, 106-108, 111, 121 and 124; and/or selected from the list consisting of SEQ ID No. 72, 74, 77-78, 80, 82, 84, 86, 88, 102, 106, 109, 114, 118, 123 and 127; and/or selected from the list consisting of SEQ ID No. 65, 70-71, 73, 80, 90, 101, 103, 105, 108, 111-112, 117, 121, 124 and 126; and/or selected from the list consisting of SEQ ID No. SEQ ID No. 69, 70, 74, 77, 84, 86, 88, 100, 108, 109, 114 and 124.

9. The LoxPsym site of claim 1, wherein the LoxPsym site is comprised in a vector.

10. The LoxPsym site of claim 1, wherein the LoxPsym site is comprised in a host cell.

11. LoxPsym site of claim 10, wherein the host cell is a plant cell, a microorganism, an insect cell or a mammalian cell.

12. LoxPsym site of claim 10, wherein the host cell is a yeast cell or a bacterial cell.

13. LoxPsym site of claim 10, wherein the host cell is a cell of a species selected form the group consisting of Saccharomyces cerevisiae, Escherichia coli, Zea mays, and Yarrowia lipolytica.

14. The LoxPsym site of claim 1, wherein the LoxPsym site forms a portion of set of at least two LoxPsym sites of claim 1, the having a first LoxPsym site and a second LoxPsym site;
wherein the first LoxPsym site and the second LoxPsym site comprise a different nucleotide on position 2, 3, 6 and/or 7 of the spacer, and wherein the set does not comprise the LoxPsym sites depicted in SEQ ID Nos. 88 and 89, SEQ ID Nos. 88 and 81, SEQ ID Nos. 90 and 83, SEQ ID Nos. 65 and 92, or SEQ ID Nos. 78 and 103.

15. The LoxPysm site of claim 14, wherein the first and second LoxPsym sites are selected from the group consisting of SEQ ID No. 65, 69-71, 74, 77, 82, 84, 100, 105, 107-109, 112, 121 and 124; and/or selected from the list consisting of SEQ ID No. 70, 77, 82, 101-102, 105, 107, 109, 112, 114, 117-118, 123-124, 126 and 127; and/or selected from the list consisting of SEQ ID No. 65, 71-74, 84, 86, 88, 90, 103, 106-108, 111, 121 and 124; and/or selected from the list consisting of SEQ ID No. 72, 74, 77-78, 80, 82, 84, 86, 88, 102, 106, 109, 114, 118, 123 and 127; and/or selected from the list consisting of SEQ ID No. 65, 70-71, 73, 80, 90, 101, 103, 105, 108, 111-112, 117, 121, 124 and 126; and/or selected from the list consisting of SEQ ID No. SEQ ID No. 69, 70, 74, 77, 84, 86, 88, 100, 108, 109, 114 and 124.

16. (canceled)

17. (canceled)

18. (canceled)

19. A method for obtaining a recombinant nucleic acid molecule, the method comprising the steps of:
a. providing a nucleic acid molecule comprising two or more nucleic acid elements each flanked by an orthogonal LoxPsym site or providing a first and a second nucleic acid molecule each comprising one or more nucleic acid elements, the one or more nucleic acid elements each flanked by an orthogonal LoxPsym site;
b. reacting the nucleic acid molecule or the first and second nucleic acid molecules with recombinase Cre to obtain the recombinant nucleic acid molecule,
wherein the orthogonal LoxPsym site is selected from the list group consisting of SEQ ID Nos. 65-127.

20. (canceled)

21. (canceled)

22. (canceled)

23. (canceled)

24. (canceled)

25. A method of optimizing gene expression of one or more genes in a cell, the method comprising the steps of:
a. introducing into a cell, one or more vectors comprising the one or more genes, each gene being under control of a promoter, the promoter comprising two or more promoter elements, wherein the two or more promoter elements are individually flanked by an orthogonal LoxPsym site, wherein each of the one or more genes comprises a different LoxPsym site;
b. expressing in said cell a recombinase Cre;
c. analysing the gene expression of the one or more genes or analysing the phenotype of the cell.

26. The method according to claim 25, wherein at least one orthogonal LoxPsym site is selected from the group consisting of SEQ ID Nos. 65-127.

27. (canceled)

28. (canceled)

29. The method according to claim 25,
further comprising the step of determining the sequence of the whole or part of the cell's genome.

30. (canceled)

31. (canceled)

32. (canceled)

33. The method according to claim 25, wherein the one or more vectors further comprise a terminator sequence downstream of each gene, the terminator sequence comprising two or more terminator elements, wherein the two or more terminator elements are individually flanked by an orthogonal LoxPsym site, wherein each of the one or more genes comprises a different LoxPsym site and wherein any of the LoxPsym sites flanking the terminator elements are different from those flanking the promoter elements.


SEQUENCE LISTING (138 sequences)

SEQ ID NO: 1-64 - skipped (64 entries)

SEQ ID NO: 65 - DNA, 34 bp, synthetic construct
  source 1..34: mol_type = other DNA
ataacttcgtatagagtactctatacgaagttat

SEQ ID NO: 66 - DNA, 34 bp, synthetic construct
  source 1..34: mol_type = other DNA
ataacttcgtataagctagcttatacgaagttat

SEQ ID NO: 67 - DNA, 34 bp, synthetic construct
  source 1..34: mol_type = other DNA
ataacttcgtatactatatagtatacgaagttat

SEQ ID NO: 68 - DNA, 34 bp, synthetic construct
  source 1..34: mol_type = other DNA
ataacttcgtatactttaaagtatacgaagttat

SEQ ID NO: 69 - DNA, 34 bp, synthetic construct
  source 1..34: mol_type = other DNA
ataacttcgtataagatatcttatacgaagttat

SEQ ID NO: 70 - DNA, 34 bp, synthetic construct
  source 1..34: mol_type = other DNA
ataacttcgtatattatataatatacgaagttat

SEQ ID NO: 71 - DNA, 34 bp, synthetic construct
  source 1..34: mol_type = other DNA
ataacttcgtatagattaatctatacgaagttat

SEQ ID NO: 72 - DNA, 34 bp, synthetic construct
  source 1..34: mol_type = other DNA
ataacttcgtataggatatcctatacgaagttat

SEQ ID NO: 73 - DNA, 34 bp, synthetic construct
  source 1..34: mol_type = other DNA
ataacttcgtatagcttaagctatacgaagttat

SEQ ID NO: 74 - DNA, 34 bp, synthetic construct
  source 1..34: mol_type = other DNA
ataacttcgtataggttaacctatacgaagttat

SEQ ID NO: 75 - DNA, 34 bp, synthetic construct
  source 1..34: mol_type = other DNA
ataacttcgtatactctagagtatacgaagttat

SEQ ID NO: 76 - DNA, 34 bp, synthetic construct
  source 1..34: mol_type = other DNA
ataacttcgtataacatatgttatacgaagttat

SEQ ID NO: 77 - DNA, 34 bp, synthetic construct
  source 1..34: mol_type = other DNA
ataacttcgtatatcatatgatatacgaagttat

SEQ ID NO: 78 - DNA, 34 bp, synthetic construct
  source 1..34: mol_type = other DNA
ataacttcgtatagtgtacactatacgaagttat

SEQ ID NO: 79 - DNA, 34 bp, synthetic construct
  source 1..34: mol_type = other DNA
ataacttcgtataacctaggttatacgaagttat

SEQ ID NO: 80 - DNA, 34 bp, synthetic construct
  source 1..34: mol_type = other DNA
ataacttcgtataatttaaattatacgaagttat

SEQ ID NO: 81 - DNA, 34 bp, synthetic construct
  source 1..34: mol_type = other DNA
ataacttcgtataatatatattatacgaagttat

SEQ ID NO: 82 - DNA, 34 bp, synthetic construct
  source 1..34: mol_type = other DNA
ataacttcgtatatcttaagatatacgaagttat

SEQ ID NO: 83 - DNA, 34 bp, synthetic construct
  source 1..34: mol_type = other DNA
ataacttcgtataagttaacttatacgaagttat

SEQ ID NO: 84 - DNA, 34 bp, synthetic construct
  source 1..34: mol_type = other DNA
ataacttcgtataggctagcctatacgaagttat

SEQ ID NO: 85 - DNA, 34 bp, synthetic construct
  source 1..34: mol_type = other DNA
ataacttcgtataaagtactttatacgaagttat

SEQ ID NO: 86 - DNA, 34 bp, synthetic construct
  source 1..34: mol_type = other DNA
ataacttcgtatagtctagactatacgaagttat

SEQ ID NO: 87 - DNA, 34 bp, synthetic construct
  source 1..34: mol_type = other DNA
ataacttcgtatattttaaaatatacgaagttat

SEQ ID NO: 88 - DNA, 34 bp, synthetic construct
  source 1..34: mol_type = other DNA
ataacttcgtatagggtaccctatacgaagttat

SEQ ID NO: 89 - DNA, 34 bp, synthetic construct
  source 1..34: mol_type = other DNA
ataacttcgtataaactagtttatacgaagttat

SEQ ID NO: 90 - DNA, 34 bp, synthetic construct
  source 1..34: mol_type = other DNA
ataacttcgtatagaatattctatacgaagttat

SEQ ID NO: 91 - DNA, 34 bp, synthetic construct
  source 1..34: mol_type = other DNA
ataacttcgtatacgttaacgtatacgaagttat

SEQ ID NO: 92 - DNA, 34 bp, synthetic construct
  source 1..34: mol_type = other DNA
ataacttcgtataaaatattttatacgaagttat

SEQ ID NO: 93 - DNA, 34 bp, synthetic construct
  source 1..34: mol_type = other DNA
ataacttcgtataaggtaccttatacgaagttat

SEQ ID NO: 94 - DNA, 34 bp, synthetic construct
  source 1..34: mol_type = other DNA
ataacttcgtataacgtacgttatacgaagttat

SEQ ID NO: 95 - DNA, 34 bp, synthetic construct
  source 1..34: mol_type = other DNA
ataacttcgtataccttaaggtatacgaagttat

SEQ ID NO: 96 - DNA, 34 bp, synthetic construct
  source 1..34: mol_type = other DNA
ataacttcgtatactgtacagtatacgaagttat

SEQ ID NO: 97 - DNA, 34 bp, synthetic construct
  source 1..34: mol_type = other DNA
ataacttcgtataccatatggtatacgaagttat

SEQ ID NO: 98 - DNA, 34 bp, synthetic construct
  source 1..34: mol_type = other DNA
ataacttcgtatacgatatcgtatacgaagttat

SEQ ID NO: 99 - DNA, 34 bp, synthetic construct
  source 1..34: mol_type = other DNA
ataacttcgtataatctagattatacgaagttat

SEQ ID NO: 100 - DNA, 34 bp, synthetic construct
  source 1..34: mol_type = other DNA
ataacttcgtatacactagtgtatacgaagttat

SEQ ID NO: 101 - DNA, 34 bp, synthetic construct
  source 1..34: mol_type = other DNA
ataacttcgtatatgatatcatatacgaagttat

SEQ ID NO: 102 - DNA, 34 bp, synthetic construct
  source 1..34: mol_type = other DNA
ataacttcgtatatcgtacgatatacgaagttat

SEQ ID NO: 103 - DNA, 34 bp, synthetic construct
  source 1..34: mol_type = other DNA
ataacttcgtatagcatatgctatacgaagttat

SEQ ID NO: 104 - DNA, 34 bp, synthetic construct
  source 1..34: mol_type = other DNA
ataacttcgtatacaatattgtatacgaagttat

SEQ ID NO: 105 - DNA, 34 bp, synthetic construct
  source 1..34: mol_type = other DNA
ataacttcgtatattctagaatatacgaagttat

SEQ ID NO: 106 - DNA, 34 bp, synthetic construct
  source 1..34: mol_type = other DNA
ataacttcgtatagtatatactatacgaagttat

SEQ ID NO: 107 - DNA, 34 bp, synthetic construct
  source 1..34: mol_type = other DNA
ataacttcgtatagtttaaactatacgaagttat

SEQ ID NO: 108 - DNA, 34 bp, synthetic construct
  source 1..34: mol_type = other DNA
ataacttcgtatagcctaggctatacgaagttat

SEQ ID NO: 109 - DNA, 34 bp, synthetic construct
  source 1..34: mol_type = other DNA
ataacttcgtatataatattatatacgaagttat

SEQ ID NO: 110 - DNA, 34 bp, synthetic construct
  source 1..34: mol_type = other DNA
ataacttcgtataccctagggtatacgaagttat

SEQ ID NO: 111 - DNA, 34 bp, synthetic construct
  source 1..34: mol_type = other DNA
ataacttcgtatagactagtctatacgaagttat

SEQ ID NO: 112 - DNA, 34 bp, synthetic construct
  source 1..34: mol_type = other DNA
ataacttcgtatatggtaccatatacgaagttat

SEQ ID NO: 113 - DNA, 34 bp, synthetic construct
  source 1..34: mol_type = other DNA
ataacttcgtatacgctagcgtatacgaagttat

SEQ ID NO: 114 - DNA, 34 bp, synthetic construct
  source 1..34: mol_type = other DNA
ataacttcgtatatagtactatatacgaagttat

SEQ ID NO: 115 - DNA, 34 bp, synthetic construct
  source 1..34: mol_type = other DNA
ataacttcgtataacttaagttatacgaagttat

SEQ ID NO: 116 - DNA, 34 bp, synthetic construct
  source 1..34: mol_type = other DNA
ataacttcgtataaattaatttatacgaagttat

SEQ ID NO: 117 - DNA, 34 bp, synthetic construct
  source 1..34: mol_type = other DNA
ataacttcgtatatgctagcatatacgaagttat

SEQ ID NO: 118 - DNA, 34 bp, synthetic construct
  source 1..34: mol_type = other DNA
ataacttcgtatatactagtatatacgaagttat

SEQ ID NO: 119 - DNA, 34 bp, synthetic construct
  source 1..34: mol_type = other DNA
ataacttcgtataccgtacggtatacgaagttat

SEQ ID NO: 120 - DNA, 34 bp, synthetic construct
  source 1..34: mol_type = other DNA
ataacttcgtatacattaatgtatacgaagttat

SEQ ID NO: 121 - DNA, 34 bp, synthetic construct
  source 1..34: mol_type = other DNA
ataacttcgtatagcgtacgctatacgaagttat

SEQ ID NO: 122 - DNA, 34 bp, synthetic construct
  source 1..34: mol_type = other DNA
ataacttcgtatacagtactgtatacgaagttat

SEQ ID NO: 123 - DNA, 34 bp, synthetic construct
  source 1..34: mol_type = other DNA
ataacttcgtatatcctaggatatacgaagttat

SEQ ID NO: 124 - DNA, 34 bp, synthetic construct
  source 1..34: mol_type = other DNA
ataacttcgtatattgtacaatatacgaagttat

SEQ ID NO: 125 - DNA, 34 bp, synthetic construct
  source 1..34: mol_type = other DNA
ataacttcgtatacggtaccgtatacgaagttat

SEQ ID NO: 126 - DNA, 34 bp, synthetic construct
  source 1..34: mol_type = other DNA
ataacttcgtatatgttaacatatacgaagttat

SEQ ID NO: 127 - DNA, 34 bp, synthetic construct
  source 1..34: mol_type = other DNA
ataacttcgtatatattaatatatacgaagttat

SEQ ID NO: 128 - DNA, 34 bp, synthetic construct
  source 1..34: mol_type = other DNA
ataacttcgtataatgtacattatacgaagttat

SEQ ID NO: 129 - protein, 343 aa, Punavirus P1
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 130 - DNA, 13 bp, synthetic construct
  source 1..13: mol_type = other DNA
ataacttcgtata

SEQ ID NO: 131 - DNA, 13 bp, synthetic construct
  source 1..13: mol_type = other DNA
tatacgaagttat

SEQ ID NO: 132 - DNA, 20 bp, synthetic construct
  source 1..20: mol_type = other DNA
tggtttagtgtatgttgcgc

SEQ ID NO: 133 - DNA, 19 bp, synthetic construct
  source 1..19: mol_type = other DNA
ctaagtctgtgctccttcc

SEQ ID NO: 134 - DNA, 13 bp, synthetic construct
  source 1..13: mol_type = other DNA
tattgaagcatat

SEQ ID NO: 135 - DNA, 13 bp, synthetic construct
  source 1..13: mol_type = other DNA
atatgcttcaata

SEQ ID NO: 136 - DNA, 34 bp, synthetic construct
  source 1..34: mol_type = other DNA
ataacttcgtatataatattctatacgaagttat

SEQ ID NO: 137 - DNA, 34 bp, synthetic construct
  source 1..34: mol_type = other DNA
ataacttcgtatatagtactctatacgaagttat

SEQ ID NO: 138 - DNA, 34 bp, synthetic construct
  source 1..34: mol_type = other DNA
ataacttcgtatataatatgctatacgaagttat